(19) United States
(12) Patent Application Publication
(10) Pub. No.: US 2026/0177546 A1
(43) Pub. Date: Jun. 25, 2026

(54) IMPLICATIONS OF CXCR3 EXPRESSION ON MYELOID CELLS FOR IMMUNOTHERAPY OF CANCER AND MYELOID-MEDIATED DISEASES

(71) Applicant: The United States of America, as represented by the Secretary, Department of Health and Human, Bethesda, MD (US)

(72) Inventors: Sabina A. Kratzmeier, Olney, MD (US); Rosandra N. Kaplan, Potomac, MD (US); Cristina Contreras Burrola, Bethesda, MD (US); Catherine Hedrick, La Jolla, CA (US); Ahmad Alimadadi, La Jolla, CA (US)

(73) Assignee: The United States of America, as represented by the Secretary, Department of Health and Human, Bethesda, MD (US)

(21) Appl. No.: 19/126,866

(22) PCT Filed: Nov. 6, 2023
(86) PCT No.: PCT/US2023/078854

§ 371 Date: May 2, 2025

Related U.S. Application Data
(60) Provisional application No. 63/423,029, filed on Nov. 6, 2022.

(51) Int. Cl. G01N 33/543; A61K 35/17; A61K 40/11; A61K 40/31; A61K 40/32; A61K 40/42; A61K 45/06; A61P 35/00; C12N 5/078; C12N 5/10; G01N 33/50; G01N 33/68

(52) U.S. Cl. CPC G01N 33/54366; A61K 35/17; A61K 40/11; A61K 40/31; A61K 40/32; A61K 40/4258; A61K 45/06; A61P 35/00; C12N 5/0634; C12N 5/10; G01N 33/5005; G01N 33/6896; C12N 2510/00; G01N 2333/7158; G01N 2800/52

(57) ABSTRACT

The invention provides a method for predicting the susceptibility of a patient suffering from a cancer or a myeloid-mediated disease to immunotherapy. Furthermore, the invention provides a method for treating a patient suffering from a cancer or a myeloid-mediated disease with immunotherapy. In accordance with these embodiments, the inventive method includes assaying myeloid cells obtained from the patient prior to treatment for the expression of CXCR3. The invention also provides a genetically engineered myeloid cell (GEMy) in which the expression of CXCR3 is modulated (up- or down-regulated). The invention further provides a composition comprising the inventive GEMy and a pharmaceutically acceptable carrier. The invention also provides a method for treating a patient in need of immunotherapy comprising administering the inventive composition to a patient.

### CROSS-REFERENCE TO RELATED APPLICATIONS

This patent application claims the benefit of U.S. Provisional Patent Application No. 63/423,029, filed Nov. 6, 2022, which is incorporated by reference.

### STATEMENT REGARDING FEDERALLY SPONSORED RESEARCH OR DEVELOPMENT

This invention was made with Government support under project numbers ZIABC011332-06, ZIABC011334-10, and NIH U01 CA 5U01CA224766-03 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 13,561 Byte XML file named “769496.XML,” dated Nov. 6, 2023.

### BACKGROUND OF THE INVENTION

Myeloid cells are a key part of the innate immune system and have the benefit of being multifunctional and show a high degree of functional plasticity in different settings. Myeloid cells are the first response to infection to clear pathogens and ramp up inflammation, are capable of antigen presentation for adaptive immune activation to orchestrate T cell-mediated and humoral immunity and are essential for wound healing by clearing dead/dying cells and for resolution of the immune response.

In cancer, myeloid cells can exhibit antitumor functions by presenting tumor antigens and producing cytokines to activate T and NK cells to direct a cytotoxic response as well as modulate the kinetics of that response. On the other hand, myeloid cells are reprogramed into a pro-tumorigenic role by the tumor to downregulate their antigen presentation machinery and produce factors that limit tumor cell killing. Myeloid cells accumulate at tumor and metastatic sites and suppress antitumor mechanisms, resulting in more aggressive cancer growth.

Immunotherapy holds promise for long-term control of cancer and other disorders. The field of immunotherapy is currently focused primarily on T cells, while myeloid cells are often overlooked. For example, studies involving chimeric antigen receptor T-cells (CAR-T cells) have revealed that myeloid cells and myeloid cell-specific gene expression programs in pre-treatment apheresis samples can inform T cell expansion and persistence following treatment. In patients receiving autologous genetically engineered CAR-T cells or T cell receptor (TCR) T cells, transferred T cells need to expand in vivo and persist long enough to infiltrate the tumor in order to be effective. In solid tumors, a major challenge is to predict which patients are more likely to have good T cell expansion and respond to T cell therapy compared to those for whom the treatment may not be beneficial. Even checkpoint inhibitors, the most common immunotherapy, can have great variability of patient responses. Also, in solid tumors, immunotherapeutic strategies have been limited by the ability of T cells to penetrate deep into and persist in tumors due to suppression by myeloid cells accumulating in the tumor microenvironment (TIE).

Taking advantage of such cells' ability to home to and infiltrate tumor and metastatic sites, Genetically Engineered Myeloid cell (GEMy) technology has been developed as a platform to locally deliver specific antitumor factors into the tumor and metastatic microenvironments, such as the TME. For example, such GEMy cells can be engineered by introduction of mRNA for fast and transient expression of cargo proteins or by lentiviral transduction for more persistent expression. GEMy cargo can include a combination of antitumor factors including but not limited to IL12, sTREM2, CD40L, IL6 decoy receptor (IL6DR), and IL1BRA.

However, a need remains for improved reagents to effectuate immunotherapy, especially, but not limited to, therapy for disorders involving solid tumors. Also, given patients' mixed responses to immunotherapies, a need remains for improved methods to assess and stratify patients in need of such therapy, which can guide clinical decision making to direct these resource-intensive cell-based and other immunotherapies to particular patients who will most likely benefit.

### BRIEF SUMMARY OF THE INVENTION

In an embodiment, the invention provides a method for predicting the susceptibility of a patient suffering from a cancer or a myeloid-mediated disease to immunotherapy. Furthermore, in an embodiment, the invention provides a method for treating a patient suffering from a cancer or a myeloid-mediated disease with immunotherapy. In accordance with these embodiments, the inventive method includes assaying myeloid cells obtained from the patient prior to treatment for the expression of at least CXCR3, and the method can involve assaying for additional markers as well, such as (but not limited to CD33, CX3CR1, CD169, HLA-DR, CD14, CD16, CD36 or CCR7).

In an embodiment, the invention also provides a genetically engineered myeloid cell (GEMy) in which the expression of CXCR3 is modulated (up- or down-regulated). In an embodiment, the inventive GEMy comprises a myeloid cell comprising an exogenous genetic expression construct comprising a cDNA or mRNA sequence encoding a CXCR3 protein (which can include an active derivative or truncated isoform thereof). In another embodiment, the inventive GEMy comprises myeloid cell genetically modified to reduce (“knock-down”) or eliminate (“knock-out”) production of a CXCR3 protein. The inventive GEMy also can be further genetically modified (such as, but not limited to, expressing other gene products).

The invention further provides a composition comprising the inventive GEMy and a pharmaceutically acceptable carrier, which can optionally include other agents in addition to the inventive GEMy and suitable carrier. The invention also provides a method for treating a patient in need of immunotherapy comprising administering the inventive composition to a patient, which can be employed as monotherapy or in connection with other therapeutic treatments of the patient.

These, and other, aspects and features of the invention will be apparent upon reading the following detailed description and reviewing the accompanying drawings.

### BRIEF DESCRIPTION OF THE SEVERAL VIEWS OF THE DRAWINGS

FIGS. 1A-1E present data demonstrating that GD2 CAR-T administration resulted in varying levels of CAR-T expansion. FIG. 1A presents a schematic of the GD2 CAR-T construct. FIG. 1B presents a timeline of treatment and sample collection for patients receiving GD2 CAR-T. FIG. 1C presents data concerning levels of CAR-T detected in the peripheral blood of patients as measured by qPCR of the GD2 CAR-T construct. FIG. 1D presents information concerning the stratification of patients into good and poor CAR-T expanders based on level of GD2 CAR-T detected by qPCR. FIG. 1E presents data concerning protein levels of cytokines in the plasma of patients 7-14 days following CAR-T administration (* p0.05; ** p0.05).

FIGS. 2A-2H present data concerning the characterization of T cells. A) Schematic of sample processing and analysis. B) UMAP clusters of immune cell populations in baseline apheresis from the T cell phenotype CyTOF panel. C) Marker gene expression in populations in baseline apheresis from the T cell phenotype CyTOF panel. D) Difference in proportion of cells in Cluster 1 between good and poor expanders. E) Flow cytometry characterization of memory CD8 and CD4 T cell populations based on CD45RA and CCR7. F) Selected C7 pathways significantly enriched in good versus poor expanders. G) PCA plot of patient samples colored by expansion. H) Heat map of top 50 differentially expressed transcription factor motifs by ATAC. sequencing.

FIGS. 3A-3F present data concerning CAR-T product and post-treatment samples display features of T cell exhaustion. A) UMAP plot of T cell populations in GD2 CAR-T product using the T cell phenotype CyTOF panel. B) Feature plots showing the distribution of expression of select markers among the T cell populations. C) Heat map of hierarchical clustering of markers in GD2 CAR-T product by CyTOF represented per patient. D) Proportion of total cells represented in clusters 3 and 6. E) CARTEx score in previously published and current datasets. F) Post-treatment sample CyTOF marker expression based on CAR-T expansion

FIGS. 4A-4D present data demonstrating that myeloid cells and myeloid cell activation programs in pre-treatment apheresis are associated with poor CAR T cell expansion. A) UMAP clusters and box plots of immune cell populations in baseline apheresis from the myeloid CyTOF panel (**** p0.0001). B) Stacked bar plots of CIBERSORT data from RNA-seq analysis delimitating predicted immune cell composition in baseline apheresis. C) Enriched gene signatures stratified by CAR-T expansion. (* p0.05). D) Selected C7 pathways significantly enriched in good versus poor expanders.

FIGS. 5A-5D present data demonstrating that CXCR3 expression on monocytes is a marker of good CAR T cell expansion. A) UMAP clusters and box plots of myeloid cell subpopulations in baseline apheresis from the myeloid CyTOF panel (* p0.05; ** p0.05; **** p0.0001). B) MDS plots of myeloid cell subpopulations in baseline apheresis labeled by patient and colored by expansion. C) Histogram plots showing expression of select markers. D) Random forest plot depicting the strength of association of markers with CAR-T expansion and representative CyTOF expression plots.

FIGS. 6A-6G present data concerning myeloid populations shift in response to CAR T cell treatment. A) Absolute monocyte count in peripheral blood 14 3 days post CAR-T infusion (* p0.05). B) Protein levels of cytokines in the plasma of patients 7-14 days and 25-27 days following CAR-T administration (* p0.05). C) Stacked bar plots of patient samples by cluster. D) Box plots of immune cell populations in pre- and post-treatment samples from the myeloid CyTOF panel. E) Box plot of CXCR3 expression on myeloid cells in pre- and post-treatment samples from the myeloid CyTOF panel. F) Overall survival of patients in TARGET-OS dataset stratified by expression level of CXCR3. G) CXCR3 expression on myeloid cells in patients hospitalized versus not hospitalized with COVID-19.

FIGS. 7A-7E present information and data concerning Cell Product Manufacturing and CAR expansion.

FIG. 8 presents data concerning cytokine analyses.

FIGS. 9A-9D present data concerning the cell phenotype across time.

FIGS. 10A-10C present data concerning the results of myeloid cell CyTOF.

FIG. 11 graphically presents a decision tree from random forest plot based on CyTOF myeloid panel Monocyte+DC subset.

FIG. 12 graphically presents data concerning CXCR3 expression on parental untransduced THP-1 cells (UTD THP-1) and on THP-1 cells engineered to overexpress CXCR3 (CXCR3+ THP-1).

FIG. 13 graphically presents data concerning CXCR3 expression on primary human monocytes and THP-1 cells following interferon treatment for 24 hours, as measured by flow cytometry.

FIG. 14 graphically presents flow cytometry data concerning the expression of CD4+ activation markers (top left panel), CD8+ activation markers (bottom left panel), CD4+ and LAG3 expression markers (top right panel), and CD8+ and LAG3 expression markers (bottom right panel) in GD2 CAR T cells co-cultured with UTD versus CXCR3-overexpressing THP-1 cells.

FIG. 15 graphically presents flow cytometry data concerning Interferon gamma production by GD2 CAR-T cells following co-culture with UTD or CXCR3+ THIP-1 cells, as measured by ELISA. P-values <0.05 are shown as analyzed by Welch t-test with Šidák's multiple comparisons test.

FIG. 16 graphically presents flow cytometry data concerning Interferon gamma production by GD2 CAR-T cells following co-culture with UTD or CXCR3+ THP-1 cells, as measured by ELISA. P-values <0.05 are shown as analyzed by Welch t-test with Šidák's multiple comparisons test.

### DETAILED DESCRIPTION OF THE INVENTION

In an embodiment, the invention provides a method for predicting the susceptibility of a patient suffering from a cancer or a myeloid-mediated disease to immunotherapy. The method also can be employed to mark a poor prognosis inflammatory state within patients. In accordance with the inventive method, myeloid cells from an apheresis product obtained from the patient are assayed prior to treatment for the expression of at least CXCR3.

In performance of the inventive method, myeloid cells to be assayed can be obtained from the patient using any standard blood collection or apheresis protocol, such as are known to those of ordinary skill in the art. In a preferred application of the method, the peripheral whole blood or apheresis product obtained from the patient can be enriched for myeloid cells (particularly dendritic cells (DC), macrophages, and monocytes) at the expense of T cells, for example, since such non-myeloid cells express CXCR3. Thus, reducing the number of lymphocytes, such as T cells, from the primary whole blood or apheresis product from the patient (or other sample or product containing myeloid cells) prior to assaying it for at least CXCR3 expression present in the lymphoid cells and thereby removing these cells can assist in removing “noise” attributed to the expression of CXCR3 within the fraction of the apheresis product otherwise represented by lymphocytes, such as T cells.

Following obtaining the whole blood or apheresis product from the patient (or other sample or product containing myeloid cells), and optionally enriching the blood or apheresis product for myeloid cells, in furtherance of the inventive method, the product containing myeloid cells is then assayed for at least the CXCR3 expression of the myeloid cells. Any suitable assay for CXCR3 expression can be employed to this end. For example, the product or sample can be analyzed by mass spectrometry, flow cytometry, immunohistochemistry, immunofluorescence staining or other suitable cytometric or immunofluorescent/immunohistochemistry protocol. In an embodiment, the assay can alternatively be performed by quantitative real time polymerase chain reaction (qRT-PCR) or enzyme-linked immunosorbent assay (ELISA).

In a preferred application of the inventive method, the assay for CXCR3 expression comprises “cytometry by time of flight” (CyTOF) analysis. While one application of CyTOF suitable for use in the inventive method is described below in Example 1, any CyTOF protocol (or, indeed, any other cytometric or other assay protocol) suitable for detecting, and preferably quantifying CXCR3 expression among myeloid cells within the blood or apheresis product (or other sample or product containing myeloid cells), can be employed in performance of the inventive method.

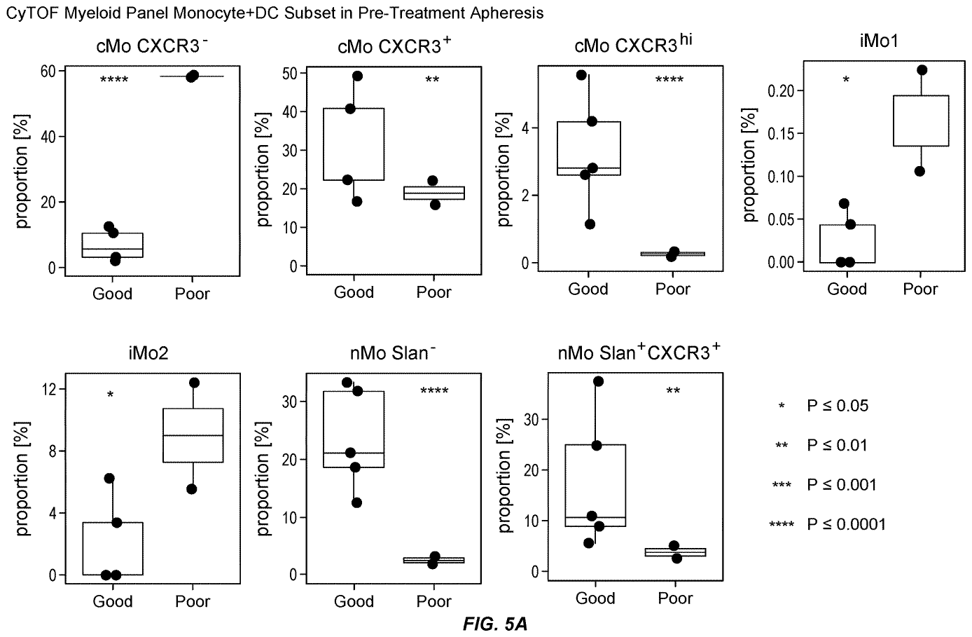

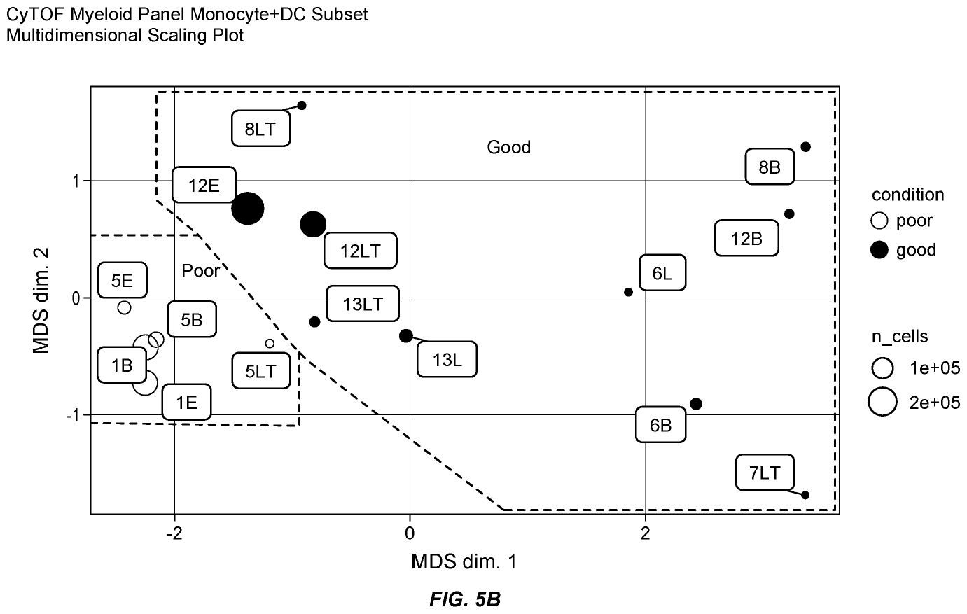

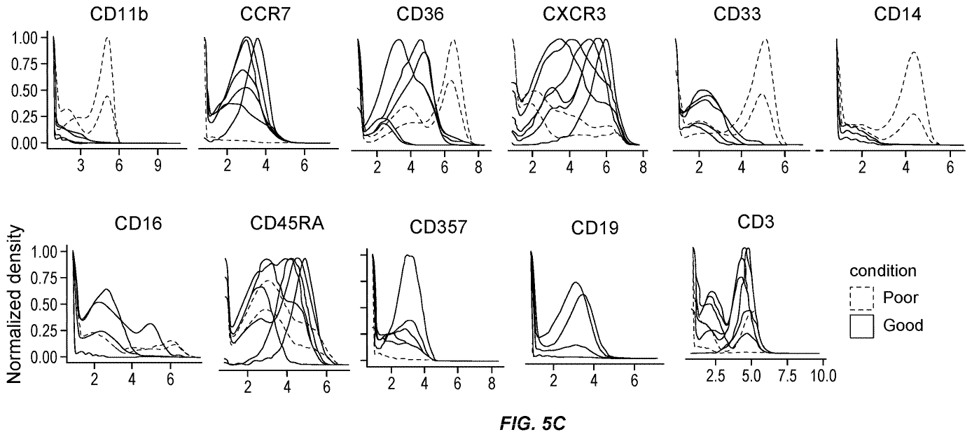

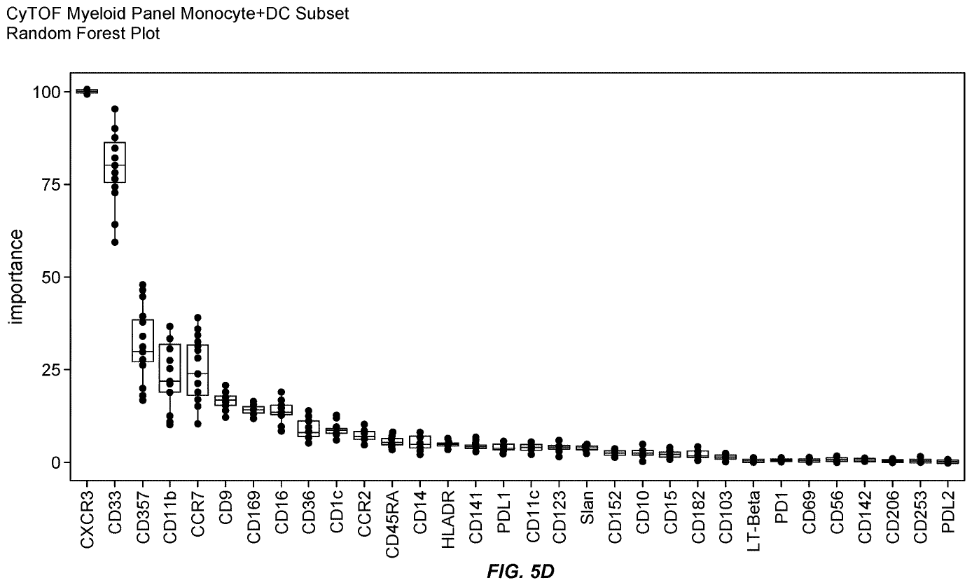

In addition to assaying for CXCR3 expression, the inventive method can also optionally include assaying the myeloid cells for additional markers as well (such as, but certainly not limited to CD33, CD169, CX3CR1, HLA-DR, CD14, CD16, CD36 or CCR7 (or a combination thereof)). For example, the results of Example 1 (See FIG. 5D) reveal that CXCR3 on myeloid cells is identified as the most robust marker of good CAR expansion. In contrast, patients who experienced poor CAR T cell expansion had increased expression of CD11b, CD36, CD33, and CD14 and low to no expression of CXCR3 in their myeloid cell populations. Random forest analysis showed that CXCR3 is the most robust marker of good CAR expansion (FIG. 5D). In contrast, patients who experienced poor CAR T cell expansion had increased expression of canonical monocyte markers CD11b and CD14, CD169, CD33, which is often used to identify MDSCs, in addition to the fatty acid transporter CD36, in their myeloid cell populations. Non-classical monocytes (CD14 negative CD16 positive, also presumably CX3CR1 positive) are associated with good expansion. CCR7 is associated with poor prognosis.

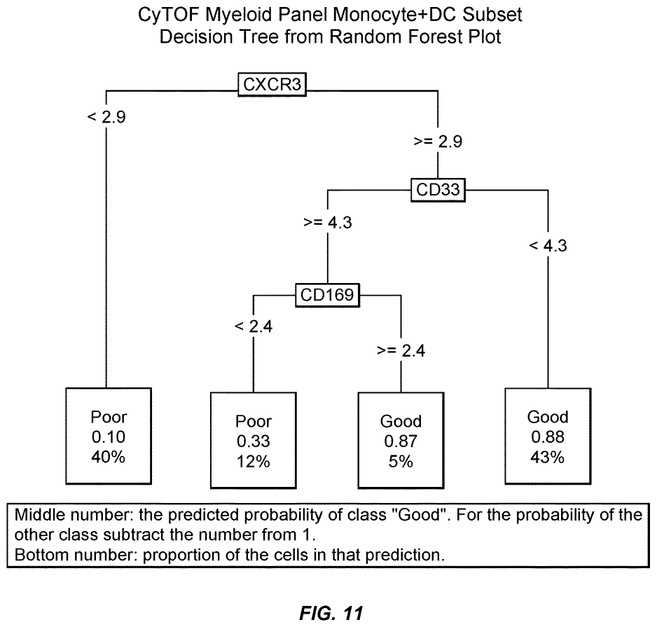

The results of the assay for at least CXCR3 expression of the myeloid cells, and optionally the assay for other markers such as, but not limited to, CD33, CD169, CX3CR1, CD16, CCR7 (or a combination thereof) then can inform clinical decision making. For example, the level of CXCR3 expression within the myeloid cells within the blood or apheresis product (or other sample or product containing myeloid cells) collected from a patient, who is a candidate for immunotherapy, can be compared to a control value resulting from a like assay for CXCR3 expression within the myeloid cells within the blood or apheresis product (or other sample or product containing myeloid cells) collected from a healthy donor or a panel of myeloid cells pooled from the blood or apheresis products (or other sample or product containing myeloid cells) collected from healthy donors. In such an application, the presence of elevated CXCR3 expression within the myeloid cells obtained from the patient relative to the control is predictive of patients that will be responsive to immunotherapy mark a good prognosis inflammatory state. Similarly, positive CXCR3 expression within the myeloid cells within the blood or apheresis product collected from the patient, coupled with the positive expression of other markers (such as, but not limited to one or more of CD33, CD169, CX3CR1, CD16, CCR7 (or a combination thereof)) likewise can be used to identify patients that are likely to be responsive to immunotherapy or mark a poorer prognosis inflammatory state. One example of the application of the inventive method, in which multiple markers are employed in combination with CXCR3 expression to inform a clinical decision is presented in FIG. 11. In this figure, presenting a decision-tree from Random Forest Plot, the values represent the normalization of data transformed from CyTOF assays, but data from flow cytometry (such as fluorescence flow cytometry) can be used. Also, the values presented in this figure are exemplary, and drawn from a single cohort of patients. Thus, while useful, the 2.9 value employed in the figure is exemplary only. The importance of the Figure is that relative levels of expression of CXCR3 expression, together with other markers (here, CD33 and CD169) can be predictive.

Furthermore, in an embodiment, the invention provides a method for treating a patient suffering from a cancer or a myeloid-mediated disease with immunotherapy. In accordance with this aspect of the invention, the method involves assessing a patient by assaying for the expression of at least CXCR3 expression within myeloid cells within the whole blood or apheresis product (or other sample or product containing myeloid cells) collected from the patient, as discussed above, followed by immunotherapy. In this respect, when the result of the assay of the myeloid cells within the apheresis product reveals elevated CXCR3 expression within the myeloid cells or positive CXCR3 expression within the myeloid cells within the whole blood or apheresis product (or other sample or product containing myeloid cells) collected from the patient, coupled with the positive expression of other markers (such as, but not limited to CD33, CD169, CX3CR1, CD16, CCR7 (or a combination thereof)), then immunotherapy is administered to the patient.

In accordance with the method of treating a patient, the immunotherapy can be any suitable type selected to treat the disorder within the patient. Thus, for example, the immunotherapy can comprise the administration of both cell-based immunotherapeutic agents, such as NK cells, T cells (including Chimeric Antigen Receptor (“CAR”)-T cells, transgenic T cell Receptor (TCR) cells, and Tumor Infiltrating Lymphocytes (“TIL therapy”), other lymphocytes, or myeloid cells (e.g., GEMys), as well as other agents, such as checkpoint inhibitors or CXCR3 agonists or antagonists.

Any cell-based immunoreagents, which are known to those of skill in the art, and many of which are approved for use or in clinical trials, can be employed. Examples include, but certainly are not limited to, the administration of a chimeric antigen receptor T cell (CAR-T cell), a genetically engineered myeloid cell (GEMy), NK cells, or a combination thereof. For example, CAR-T cells expressing CD19, CBMA, and the like have been approved for clinical use, and can employed in the context of the present invention. Similarly, other CAR-T cells, such as, but not limited to, those expressing GD2, B87H3, FGFR4, CD22, Her2, and the like, also can be employed. Another type of cell-based immunoreagent that can be employed therapeutically in the context of the inventive method includes GEMys. Thus, for example, the method can involve administration of one or more GEMy that secretes “interleukin-12” (IL12), soluble “triggering receptor expressed on myeloid cells 2” (sTREM2), “cluster of differentiation 40 ligand” (CD40L), “interleukin 6 decoy receptor” (IL6DR), IL1BRA, Hyal, or a combination thereof. These genetic modifications mentioned here are by way of example only, and the invention is not limited in its therapeutic application to the use of these reagents recited here. Indeed, it is within the scope of the skill of a treating physician or other clinician to select a cell-based immunotherapy appropriate to treating a particular condition from which the patient is suffering.

Moreover, the therapeutic application of the inventive method is not limited to cell-based immunotherapies. In this respect, the immunotherapy can involve administration of immunoglobulins or similar molecules (e.g., conjugates, Fab fragments, and the like) to patients, in accordance with established protocols or as otherwise known to those of skill in the art. A specific non-limiting example of this type of therapy particularly suitable for cancer includes checkpoint inhibition, which can be achieved, for example, by administering reagents such as antibodies that target molecules such as CTLA4, PD-1, PD-L1, TIGIT, Tim3 and CD47, for example. Several such reagents have been approved for clinical use, and a skilled clinician can employ these, or other suitable checkpoint inhibitors, as desired for application to a particular patient.

In another embodiment, the invention provides a GEMy cell comprising a myeloid cell, which is genetically engineered to modulate the expression (up- or down-modulate) of a sequence encoding a CXCR3 protein. The GEMy can be a monocyte, a macrophage, or a myeloid dendritic cell or conventional dendritic cell (DC), and preferably is within a population of such cells, which can be a mixture of genetically modified monocytes, macrophages, and myeloid DCs, which are genetically modified to modulate the expression (up- or down-modulate) of a sequence encoding a CXCR3 protein. While typically, the GEMy will be derived from a human patient or donor, the animal source can be any desired species, such as for veterinary applications (cats, dogs, horses, etc.) or laboratory applications (mice, rats, non-human primates, and the like).

In the context of the present invention, a CXCR3 protein refers to any polypeptide that forms a functional CXCR3 molecule. Such polypeptides can, of course, include a naturally-occurring wild-type CXCR3 isoform, three of which (SEQ ID NOs:1-3) are reproduced below. However, the invention is not limited to polypeptides having these precise sequences but can include any functionally equivalent polypeptide. For example, particularly for embodiments in which the expression of a CXCR3 protein is up-modulated, the inventive GEMy cell can comprise an exogenous expression construct comprising a nucleic acid sequence encoding a derivative of SEQ ID NOs: 1-3, such as a truncated version of such, or a fusion protein comprising a CXCR3 protein and another moiety. Moreover, the amino acid sequence of a CXCR3 protein can vary from those presented in SEQ ID NO:s 1-3. Thus, a particular a CXCR3 protein in the context of the present invention may comprise one or more amino acid substitutions, so long as the resulting CXCR3 protein retains the function of CXCR3. To preserve proper folding and produce a functional tertiary structure functioning as a CXCR3 protein, preferably such substations comprise, but are not limited to “conservative” substitutions (e.g., in which a hydrophobic amino acid is replaced by another hydrophobic amino acid, an acidic amino acid is replaced by another acidic amino acid, a basic amino acid is replaced by another basic amino acid, etc.). In this respect, a CXCR3 protein, can be expressed within the inventive GEMy cell that bears at least 50% amino acid sequence identity with one of SEQ ID NOs:1-3, such as at least 60% amino acid sequence identity with one of SEQ ID NOs:1-3, such as at least 70% amino acid sequence identity with one of SEQ ID NOs:1-3, such as at least 80% amino acid sequence identity with one of SEQ ID NOs:1-3, such as at least 90% amino acid sequence identity with one of SEQ ID NOs:1-3, such as at least 95% amino acid sequence identity with one of SEQ ID NOs:1-3, such as at least 97% amino acid sequence identity with one of SEQ ID NOs:1-3, such as at least 99% amino acid sequence identity with one of SEQ ID NOs:1-3.

Also, in the context of the present invention, a genetic sequence encoding a CXCR3 protein can be or comprise any sequence of nucleic acids which result in the production of a functional CXCR3 protein, including wild-type CXCR3 protein isoforms (e.g., SEQ ID NOs:1-3) or derivatives or modifications from such sequences as discussed in the preceding paragraph. Exemplary cDNA/RNA sequences encoding two wild-type CXCR3 protein isoforms are set forth herein as SEQ ID NOs:4 and 5, but the invention is not limited to the use of these sequences, even for producing a wild-type CXCR3 protein. Indeed, the degeneracy of the genetic code is well-known to persons of ordinary skill in the art, and it is within the ambit of such skill to design a coding polynucleotide suitable for producing any of SEQ ID NOs:1-3 or a derivative or variant thereof, such as are described in the preceding paragraph. Moreover, the coding sequence can be or comprise a cDNA, an RNA, or genomic DNA, although for genetic manipulation of cells, cDNA sequences are most readily used to construct appropriate expression vectors.

In one embodiment, the inventive GEMy cell is engineered to up-regulate the expression of CXCR3. It is believed that such GEMy cells, expressing CXCR3, can improve homing, activation of cytotoxic T and NK cells, and provide an immune activating feed-forward loop in the development of myeloid cells for therapy. Thus, to facilitate up-regulation of CXCR3, the inventive GEMy can comprise a myeloid cell comprising an exogenous genetic expression construct comprising a genetic sequence encoding a CXCR3 protein. The addition of CXCR3 on GEMys or other myeloid or T cell cell-based therapy can impart improved homing and persistence and therefore use of CXCR3 expression in these modified cells can improve that aspect. In one embodiment, the inventive GEMy cell or other engineered cell can be exposed to ligands in manufacture to upregulate or downregulate CXCR3 expression.

The sequence encoding a CXCR3 protein can be introduced into the myeloid cell by any suitable means, such as electroporation or transfection/infection with a plasmid or viral vector. Myeloid cells can be engineered as GEMys by introduction of mRNA encoding the CXCR3 protein, for example, which provides for fast and transient expression of CXCR3 as a “cargo” protein. Alternatively, transduction with a viral vector system, such as a lentiviral vector comprising a cDNA sequence encoding the CXCR3 protein, can be employed if more persistent expression of the CXCR3 protein is desired. An example of the construction of GEMy cells is presented in Kaczanowska et al., Cell 184, 2033-2052 (2021), which is incorporated by reference in its entirety herein.

For up-modulating the expression of CXCR3 protein within the engineered inventive GEMy cells, because as noted above, myeloid cells of healthy patients typically do not express appreciably the CXCR3 protein, or express it at a very low level, preferably the nucleic sequence to be introduced into the myeloid cell, which encodes the CXCR3 protein, is under the control of a strong constitutive promoter or a myeloid-specific promoter, so as to facilitate robust expression of the CXCR3 protein within the GEMy. Suitable myeloid-specific promoters for use in this context include, but are not limited to, myeloid specific synthetic promoters (sp-144, sp-107) and myeloid specific promoters for improved expression of desired cargo proteins in myeloid cells, such as, myeloid specific transcription factor promoters SP1, Pu.1a, Pu.1b, C/EBPa, AP1, AML-1, LYSMD1, CBF, BCL2, MYB, GATA, MMP14, and variants thereof. It should be observed that the recitation of these promoters and regulatory elements is for example only, and that other myeloid-specific or constitutive promoters/enhancer elements can be selected by a person of ordinary skill in the art.

In another embodiment, the inventive GEMy cell is engineered to down-regulate the expression of CXCR3. While, as noted, expression of CXCR3 is typically low in cells from healthy individuals, it is believed that down-regulation of CXCR3 within such GEMy cells can effectively prevent myeloid infiltration in diseased individuals, in which myeloid infiltration supports disease processes such as atherosclerosis and Alzheimer's disease. Thus, in one embodiment, the invention provides a myeloid cell genetically modified to reduce or eliminate production of a CXCR3 protein.

To achieve down-modulation of the expression or production of a CXCR3 protein, the native expression of CXCR3 within the myeloid cells can be either knocked-down or knocked out. Thus, in one approach, the inventive GEMy comprises a myeloid cell comprising an exogenous genetic expression construct encoding an interfering RNA targeted to a sequence encoding a CXCR3 protein. As with the embodiment discussed above, in which exogenous coding sequences are introduced into the GEMy, so too the interfering RNA (or a sequence encoding such) can be introduced into the myeloid cells by electroporation, or transfection/infection of the cells with RNA or a vector (such as a lentiviral vector, or other suitable vector) comprising a nucleotide sequence encoding RNA sufficiently complementary to the native CXCR3 protein coding sequence to effectuate RNAi. Given that coding sequences relevant to CXCR3 protein are known (see, e.g., SEQ ID NOs: 4 and 5), those of ordinary skill in the art will be able to design an appropriate interfering RNA sequence to effectively attenuate expression of CXCR3 within the inventive GEMy.

In another approach, down-modulation of the expression or production of a CXCR3 protein is effectuated by eliminating all or a portion of a sequence encoding native CXCR3, and/or its regulatory sequence, from the chromosomal DNA of a myeloid cell. For generating such embodiments of the inventive GEMy in which the native CXCR3 is “knocked out,” techniques such as CRISPR, single base editing, the use of Transcription Activator-like Effector Nucleases (TALENs) or Zinc Finger proteins can be employed for the process of genetic manipulation of the myeloid cells.

A preferred method to generate the inventive GEMy engineered to down-regulate the expression of CXCR3 involves CRISPR. CRISPR-Cas systems employ a tracrRNA which plays a role in the maturation of crRNA. The tracrRNA is partially complementary to and base pairs with a pre-crRNA forming an RNA duplex. This is cleaved by RNase III to form a crRNA/tracrRNA hybrid, which acts as a guide for the endonuclease Cas9, which cleaves the invading nucleic acid. Typically, for CRISPR applications, the Cas9 nickase enzyme is co-transfected with a guide RNA (“gRNA”) to effectuate gene editing. However, enzymes other than Cas9 (such as, for example Cas12a) can be suitably employed in some embodiments. As information concerning the genetic sequences for CXCR3 isoforms is known, suitable gRNAs for use in targeting CRISPR-Cas9 (or other suitable CRISPR system) to knock out all or a portion of CXCR3 from myeloid cells to generate the inventive GEMy cells can readily be designed by persons of ordinary skill in the art.

CRISPR technology is well known to persons of ordinary skill in the art, and any suitable protocol can be employed in the context of the present invention. In one protocol, tracrRNA and crRNAs (respectively containing sequences targeting genomic CXCR3 coding and/or regulatory sequences) can be suspended in a buffer and incubated at 95° C. for five minutes to generate gRNA, which is thereafter incubated with Cas9 protein to generate Cas9-ribonucleotide proteins (Cas9-RNP). Such Cas9-RNP then can be mixed with naïve myeloid cells, or GEMys, and the mixture subjected to electroporation to effect nucleofection of the Cas9-RNP into the cells.

As noted, other methods for gene editing can be employed as alternatives to CRISPR to generate embodiments of the inventive GEMy in which the CXCR3 gene is “knocked out” (applicable as well for embodiments in which the inventive cell lacks functional expression of CXCR3). Such methods include those employing transcription activator like effector nucleases (TALENs) and the use of Zinc finger proteins, for example. For each application, standard methodology known to those of ordinary skill can be employed to generate the genetically altered GEMy cells of the present invention.

TALENs are customized artificial restriction nucleases that can be readily constructed to target a known genetic sequence, using methods known to persons of ordinary skill in the art. Similarly, Zinc finger domains can be engineered using methods known to persons of ordinary skill in the art to target specific desired DNA sequences, which this enables Zinc finger nucleases to target unique sequences within complex genomes to alter the chromosomal DNA of cells. Thus, knowledge of sequences for CXCR3 (for example, as set forth in SEQ ID NOs: 5 and 6 and otherwise known in the art) can facilitate the design and construction of TALENs and Zinc finger nucleases, targeting the same genome loci that CXCR3 guide RNAs bind, suitable for generating the inventive GEMy cells with reduced or diminished CXCR3 expression (lacking functional FIBP and/or TMEM222 expression) in which CXCR3 is “knocked out.”

It should be understood that, in addition to having a modulated expression of CXCR3 (either up- or down-modulated) as described above, the inventive GEMy cell also can include additional genetic modifications. For example, such cells can be engineered to also express other exogenous genetic material, such as to produce soluble factors, such as IL12, sTREM2, CD40L, IL6 decoy receptor (IL6DR), IL1BRA, among others.

Following prediction, the inventive GEMy cell or cells, i.e., modified to up- or down-modulate the expression of a CXCR3 protein, can be expanded into a population of such cells, which can be homogenous or inhomogeneous (heterogeneous) (e.g., comprising several types of myeloid cells (e.g., a mixture of monocytes, macrophages, and DCs). Such cells or populations thereof then can be formulated into formulations suitable for therapeutic use.

Thus, in an embodiment, the invention provides a composition comprising the GEMy having modulated expression of a CXCR3 protein as described herein (or a population thereof) and a carrier. The composition comprising the inventive the GEMy having modulated expression of a CXCR3 protein can comprise a suitable carrier which is physiologically compatible with the inventive GEMy cells, which is taken to mean that the carrier is able to maintain the viability of the inventive GEMy cells until the point at which the composition is to be used. In some embodiments, the carrier can be a suitable culture medium to support continued maintenance and expansion of the inventive GEMy cells. In an embodiment, the composition is frozen (e.g., lyophilized), and can be thawed and reconstituted prior to use. In an embodiment, the carrier of a component of the composition can be a pharmaceutically acceptable carrier; accordingly, the invention provides a pharmaceutical composition comprising the inventive GEMy cells having modulated expression of a CXCR3 protein and a pharmaceutically acceptable carrier.

The pharmaceutical composition of the present invention, whether comprising the inventive GEMy cells or other agents, can be formulated for any desired mode of administration (e.g., as a solution, implantable structure, salve, etc.). A preferred formulation includes a liquid carrier, which facilitates administration to a patient via injection (such as intravenously, interperitoneally, intramuscularly, or by intratumoral injection, for example). A suitable carrier for injection can comprise sterile saline and can include excipients and adjuvants known to persons of ordinary skill, such as buffers, growth factors, preservatives, and the like, to facilitate storage and maintain the viability of the inventive GEMy cells within the pharmaceutical composition.

Within the inventive composition, the inventive GEMys can be present in any desired concentration or titer. However, a suitable dosage can include the inventive GEMys in an amount sufficient to deliver between about 1×10⁵ cells/kg to about 1×10⁹ cells/kg to a human patient.

The pharmaceutical composition of the present invention also can include active agents in addition to the inventive GEMy cells having modulated expression of a CXCR3 protein, such as chemotherapeutic agents, antibody therapies, lymphocytes, myeloid cells, NK cells, peptide vaccines, RNA vaccines, immune adjuvants, and other agents, which are offered here purely as non-limiting examples. Likewise, the inventive composition can be employed therapeutically in connection with other compositions comprising such agents.

For example, the inventive pharmaceutical composition can include, or be administered in conjunction with, agents such as chemotherapeutic agents. Examples of such chemotherapeutic agents for use in the context of the invention include, but certainly are not limited to, agents such as cyclophosphamide, fludarabine, or a combination thereof.

Also, suitable agents to be included in the inventive composition together with the inventive GEMy cells having modulated expression of a CXCR3 protein, or to be administered in conjunction with the inventive composition, include therapeutic antibodies. Examples of such therapeutic antibodies for use in the context of the invention include tumor-targeting and immune checkpoint antibody therapies, bispecific antibodies, antibody drug conjugates, and the like, or combinations thereof.

Also, suitable agents to be included in the inventive composition together with the inventive GEMy cells having modulated expression of a CXCR3 protein, or to be administered in conjunction with the inventive composition, include cell-based therapies, such as lymphocytes, myeloid cells, NK cells, and the like, which can be genetically modified. Thus, for example, which is in no way intended to limit the scope of the invention, the inventive composition can include, in addition to the inventive GEMy cells having modulated expression of a CXCR3 protein, lymphocytes, which can be conditioned using standard methods or engineered for adoptive transfer or be administered in conjunction with such agent. Such cells include, for example, conditioned or engineered natural killer (NK) cells or T cells, and the use of such cells (e.g., tumor infiltrating lymphocytes, transgenic T cell receptor T cells, CAR-T cells, or combinations thereof) in conjunction with the inventive GEMy cells having modulated expression of a CXCR3 protein is contemplated. Thus, for example, CD19, CBMA, and the like have been approved for clinical use, and can employed in the context of the present invention. Similarly, other CAR-T cells, such as, but not limited to, those expressing GD2, B87H3, FGFR4, CD22, Her2, and the like, also can be employed in conjunction with the inventive GEMy cells having modulated expression of a CXCR3 protein.

By way of another example, which is by no way intended to limit the scope of the invention, the inventive composition can include, in addition to the inventive GEMy cells having modulated expression of a CXCR3 protein, a myeloid cell, such as one or more GEMys (e.g., a modified DC, monocyte, or dendritic cell).

Other examples of agents that can optionally be included within the inventive composition, or administered in conjunction with it to effect therapy, include peptide or RNA agents targeting cancers. The composition also can include, or be administered in conjunction with, immune adjuvants, such as but not limited to, a STING agonist, a Toll-like receptor agonist, and the like, or any suitable combination thereof.

The inventive pharmaceutical composition can be manufactured according to standard methodology, which, in respect of the inventive GEMy cells, involves suspending the inventive GEMy cells in the desired carrier, under sterile conditions and desirably using Good Manufacturing Practices (GMP). Other agents for inclusion in the inventive composition, or any component thereof, can be formulated using methods known to those of skill in the art. Also, inventive composition can be packaged in a suitable manner to facilitate its use, such as in vials, ampoules, syringes, and the like.

It will be observed that the inventive composition can be used therapeutically. Thus, in an embodiment, the invention provides a method for treating a patient in need of immunotherapy comprising administering the inventive composition as described herein, i.e., containing the inventive GEMy cell or cells having modulated expression of a CXCR3 protein, and optionally other agents. The composition is administered in an amount and at a location to impart a therapeutic effect in the patient.

For treatment in accordance with the methods of the present invention, either involving administering the inventive composition comprising the inventive GEMy cell or cells having modulated expression of a CXCR3 protein or the inventive method, as discussed immediately above, or the embodiment involving assessing a patient by assaying for the expression of at least CXCR3 expression within myeloid cells within the apheresis, whole blood or other product collected from the patient followed by immunotherapy, also as discussed above, and also in its diagnostic application, the patient can be any patient amenable to immunotherapy, which can be cell-based or molecular immunotherapy. The inventive therapeutic methods, thus, are employed therapeutically to treat the patient suffering from such a disease or disorder amenable to immunotherapy or biological therapy. For example, the patient can be suffering from (and the inventive methods employed to treat) conditions such as cancers, autoimmune disease or disorder, inflammatory diseases or disorders, degenerative diseases or disorders, and infectious disease, among others.

For example, the patient can be suffering from (and the inventive methods employed to treat) cancers such as sarcomas, neuroblastoma, neuroendocrine tumors, breast cancer, pancreatic adenocarcinoma, ovarian cancer, and melanoma, although these are intended as non-limiting examples; the invention can be used to treat a wide variety of other cancer types as well. The method is particularly suitable to treatment of cancers manifesting as solid tumors, which is particularly useful given the resistance of such tumors to many commonly-employed immunotherapies.

In other embodiments, in accordance with the diagnostic and therapeutic applications of the present invention, the patient can be suffering from (and the inventive methods employed to treat) an autoimmune disease or disorder. Non-limiting examples of such disorders include, but are not limited to for example, inflammatory bowel disease (IBD), rheumatoid arthritis, plaque psoriasis, lupus, graft versus host disease (GVHD), and the like, although the inventive methods can be useful in the context of other autoimmune diseases or disorders as well.

In other embodiments, in accordance with the diagnostic and therapeutic applications of the present invention, the patient can be suffering from (and the inventive methods employed to treat) an inflammatory disease or disorder. Non-limiting examples of such disorders include, but are not limited to sepsis, atherosclerosis, and the like, although the inventive methods can be useful in the context of other inflammatory diseases or disorders as well.

In other embodiments, in accordance with the diagnostic and therapeutic applications of the present invention, the patient can be suffering from (and the inventive methods employed to treat) a degenerative disease or disorder. Non-limiting examples of such disorders include, but are not limited to, those involving neurodegeneration, such as, for example, Alzheimer's Disease, although the inventive methods can be useful in the context of other degenerative diseases or disorders as well.

In other embodiments, in accordance with the diagnostic and therapeutic applications of the present invention, the patient can be suffering from (and the inventive methods employed to treat) am infectious disease. Non-limiting examples of such infectious diseases include, but are not limited to, those involving viral or bacterial infections, such as SARS-CoV-2 (COVID). However, although the inventive methods can be useful in the context of other infectious diseases as well.

Also, the inventive therapeutic methods can be employed alone in the treatment of a patient or employed in connection with another therapy. Thus, for example, the inventive methods can be employed in conjunction with methods involving chemotherapy, radiation therapy, surgery, antibody therapy, adoptive transfer of lymphocytes, dendritic cell or other myeloid cell therapies, peptide or RNA cancer vaccines, immune adjuvants, or a combination thereof.

Furthermore, in the context of the present description of the invention, a “patient” can be any individual suffering from an applicable disease or disorders, as discussed herein. Typically, the patient will be a human being, and can be juvenile or adult. However, the therapeutic and diagnostic methods disclosed herein also have utility in veterinary and laboratory settings. Thus, in context, a “patient” can be a companion animal or livestock (e.g., a dog, a housecat, a horse) or an animal of zoological interest (e.g., large cats, ungulates, elephants, cetaceans, etc.). Also, for laboratory use, the method can be employed using “patients” drawn from species, especially mammalian species, typical for laboratory use, such as mice, rats, non-human primates, and the like.

### Sequences

The following biological sequences are referenced herein:

SEQ ID NO: 1 Homo sapiens Isoform 1 Amino Acid Sequence: CXCR3-A
MVLEVSDHQVLNDAEVAALLENFSSSYDYGENESDSCCTSPPCPQDFSLNFDRAFLPAL
YSLLFLLGLLGNGAVAAVLLSRRTALSSTDTFLLHLAVADTLLVLTLPLWAVDAAVQW
VFGSGLCKVAGALFNINFYAGALLLACISFDRYLNIVHATQLYRRGPPARVTLTCLAVW
GLCLLFALPDFIFLSAHHDERLNATHCQYNFPQVGRTALRVLQLVAGFLLPLLVMAYCY
AHILAVLLVSRGQRRLRAMRLVVVVVVAFALCWTPYHLVVLVDILMDLGALARNCGR
ESRVDVAKSVTSGLGYMHCCLNPLLYAFVGVKFRERMWMLLLRLGCPNQRGLQRQPS
SSRRDSSWSETSEASYSGL
SEQ ID NO: 2 Homo sapiens Isoform 2 Amino Acid Sequence: CXCR3-B
MELRKYGPGRLAGTVIGGAAQSKSQTKSDSITKEFLPGLYTAPSSPFPPSQVSDHQVLND
AEVAALLENFSSSYDYGENESDSCCTSPPCPQDFSLNFDRAFLPALYSLLFLLGLLGNGA
VAAVLLSRRTALSSTDTFLLHLAVADTLLVLTLPLWAVDAAVQWVFGSGLCKVAGALF
NINFYAGALLLACISFDRYLNIVHATQLYRRGPPARVTLTCLAVWGLCLLFALPDFIFLS
AHHDERLNATHCQYNFPQVGRTALRVLQLVAGFLLPLLVMAYCYAHILAVLLVSRGQR
RLRAMRLVVVVVVAFALCWTPYHLVVLVDILMDLGALARNCGRESRVDVAKSVTSGL
GYMHCCLNPLLYAFVGVKFRERMWMLLLRLGCPNQRGLQRQPSSSRRDSSWSETSEAS
YSGL
SEQ ID NO: 3 Homo sapiens Isoform 3 Amino Acid Sequence: CXCR3-alt
MVLEVSDHQVLNDAEVAALLENFSSSYDYGENESDSCCTSPPCPQDFSLNFDRAFLPAL
YSLLFLLGLLGNGAVAAVLLSRRTALSSTDTFLLHLAVADTLLVLTLPLWAVDAAVQW
VFGSGLCKVAGALFNINFYAGALLLACISFDRYLNIVHATQLYRRGPPARVTLTCLAVW
GLCLLFALPDFIFLSAHHDERLNATHCQYNFPQGSSSGSGCGCCSCAWAAPTREGSRGS
HRLPAGIHPGLRPQRPPTRACEAGIRAPLSPI
SEQ ID NO: 4 Homo sapiens chemokine receptor 3 isoform 1 (CXCR3) mRNA,
partial cds GenBank: KU178101.1
>KU178101.1 Homo sapiens chemokine receptor 3 isoform 1 (CXCR3) mRNA,
partial cds
CCGCCCTCACAGGTGAGTGACCACCAAGTGCTAAATGACGCCGAGGTTGCCGCCCT
CCTGGAGAACTTCAGCTCTTCCTATGACTATGGAGAAAACGAGAGTGACTCGTGCTG
TACCTCCCCGCCCTGCCCACAGGACTTCAGCCTGAACTTCGACCGGGCCTTCCTGCC
AGCCCTCTACAGCCTCCTCTTTCTGCTGGGGCTGCTGGGCAACGGCGCGGTGGCAGC
CGTGCTGCTGAGCCGGCGGACAGCCCTGAGCAGCACCGACACCTTCCTGCTCCACCT
AGCTGTAGCAGACACGCTGCTGGTGCTGACACTGCCGCTCTGGGCAGTGGACGCTG
CCGTCCAGTGGGTCTTTGGCTCTGGCCTCTGCAAAGTGGCAGGTGCCCTCTTCAACA
TCAACTTCTACGCAGGAGCCCTCCTGCTGGCCTGCATCAGCTTTGACCGCTACCTGA
ACATAGTTCATGCCACCCAGCTCTACCGCCGGGGGCCCCCGGCCCGCGTGACCCTCA
CCTGCCTGGCTGTCTGGGGGCTCTGCCTGCTTTTCGCCCTCCCAGACTTCATCTTCCT
GTCGGCCCACCACGACGAGCGCCTCAACGCCACCCACTGCCAATACAACTTCCCAC
AGGTGGGCCGCACGGCTCTGCGGGTGCTGCAGCTGGTGGCTGGCTTTCTGCTGCCCC
TGCTGGTCATGGCCTACTGCTATGCCCACATCCTGGCCGTGCTGCTGGTTTCCAGGG
GCCAGCGGCGCCTGCGGGCCATGCGGCTGGTGGTGGTGGTCGTGGTGGCCTTTGCCC
TCTGCTGGACCCCCTATCACCTGGTGGTGCTGGTGGACATCCTCATGGACCTGGGCG
CTTTGGCCCGCAACTGTGGCCGAGAAAGCAGGGTAGACGTGGCCAAGTCGGTCACC
TCAGGCCTGGGCTACATGCACTGCTGCCTCAACCCGCTGCTCTATGCCTTTGTAGGG
GTCAAGTTCCGGGAGCGGATGTGGATGCTGCTCTTGCGCCTGGGCTGCCCCAACCAG
AGAGGGCTCCAGAGGCAGCCATCGTCTTCCCGCCGGGATTCATCCTGGTCTGAGACC
TCAGAGGCCTCCTACTCGGGCTTG
SEQ ID NO: 5 Homo sapiens chemokine receptor 3 isoform 2 (CXCR3) mRNA,
partial cds, alternatively spliced
GenBank: KU178102.1
>KU178102.1 Homo sapiens chemokine receptor 3 isoform 2 (CXCR3) mRNA,
partial cds, alternatively spliced
CCGCCCTCACAGGTGAGTGACCACCAAGTGCTAAATGACGCCGAGGTTGCCGCCCT
CCTGGAGAACTTCAGCTCTTCCTATGACTATGGAGAAAACGAGAGTGACTCGTGCTG
TACCTCCCCGCCCTGCCCACAGGACTTCAGCCTGAACTTCGACCGGGCCTTCCTGCC
AGCCCTCTACAGCCTCCTCTTTCTGCTGGGGCTGCTGGGCAACGGCGCGGTGGCAGC
CGTGCTGCTGAGCCGGCGGACAGCCCTGAGCAGCACCGACACCTTCCTGCTCCACCT
AGCTGTAGCAGACACGCTGCTGGTGCTGACACTGCCGCTCTGGGCAGTGGACGCTG
CCGTCCAGTGGGTCTTTGGCTCTGGCCTCTGCAAAGTGGCAGGTGCCCTCTTCAACA
TCAACTTCTACGCAGGAGCCCTCCTGCTGGCCTGCATCAGCTTTGACCGCTACCTGA
ACATAGTTCATGCCACCCAGCTCTACCGCCGGGGGCCCCCGGCCCGCGTGACCCTCA
CCTGCCTGGCTGTCTGGGGGCTCTGCCTGCTTTTCGCCCTCCCAGACTTCATCTTCCT
GTCGGCCCACCACGACGAGCGCCTCAACGCCACCCACTGCCAATACAACTTCCCAC
AGGGGTCAAGTTCCGGGAGCGGATGTGGATGCTGCTCTTGCGCCTGGGCTGCCCCAA
CCAGAGAGGGCTCCAGAGGCAGCCATCGTCTTCCCGCCGGGATTCATCCTGGTCTGA
GACCTCAGAGGCCTCCTACTCGGGCTTG

| Target | Primer/Probe Name | Sequence (5′-3′) |
|---|---|---|
| CDKN1A | Sense primer (SEQ ID NO: 6) | GAAAGCTGACTGCCCCTATTTG |
| | Antisense primer | GAGAGGAAGTGCTGGGAACAAT |
| | (SEQ ID NO: 7) | |
| | Probe (SEQ ID NO: 8) | CTCCCCAGTCTCTTT |
| OX40Z | Sense primer (SEQ ID NO: 9) | CGCCCACTCCACCCT |
| | Antisense primer | GTTCTGGCCCTGCTGGTA |
| | (SEQ ID NO: 10) | |
| | Probe (SEQ ID NO: 11) | CAAGATCAGAGTGAAGTTC |

### EXAMPLES

These experimental Examples are presented here to further illustrate the invention but, of course, should not be construed as in any way limiting its scope. Among other findings, the data presented in these Examples demonstrate that CXCR3 expression on peripheral monocytes at baseline is associated with improved CAR-T cell expansion in vivo.

### Example 1

This Example is based on a Phase 1 dose escalation trial of GD2 CAR-Ts in children and young adults with GD2+ solid tumors, nine of which had been diagnosed with osteosarcoma and two with neuroblastoma.

### Methods

### Trial Design

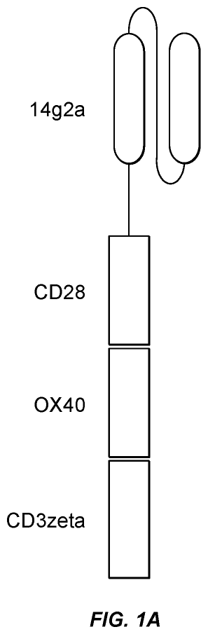

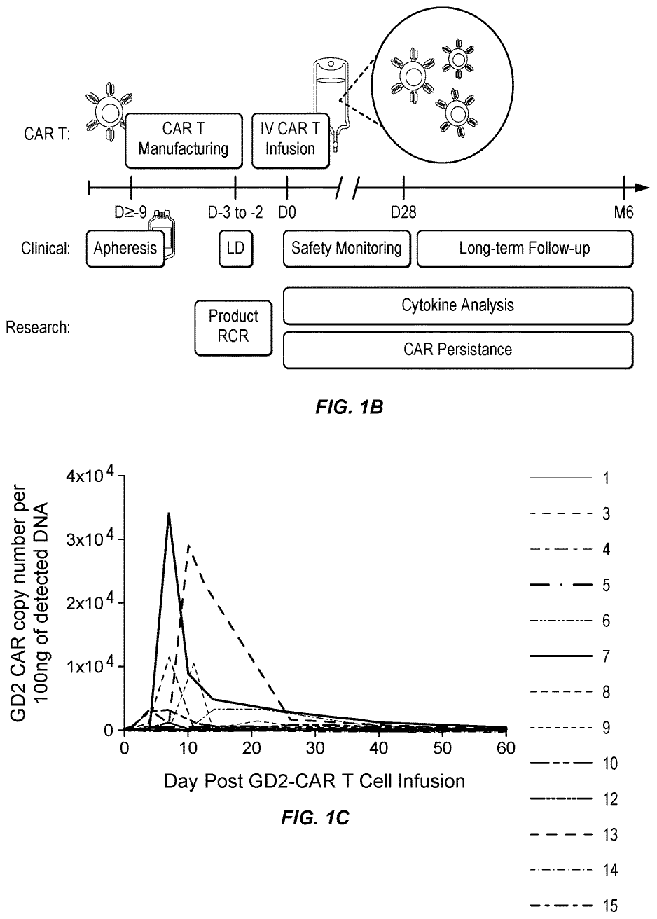

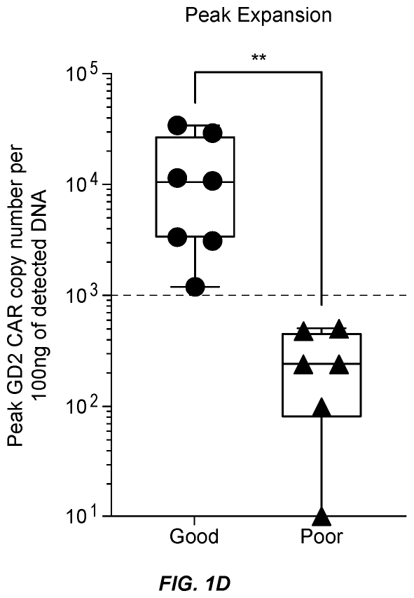

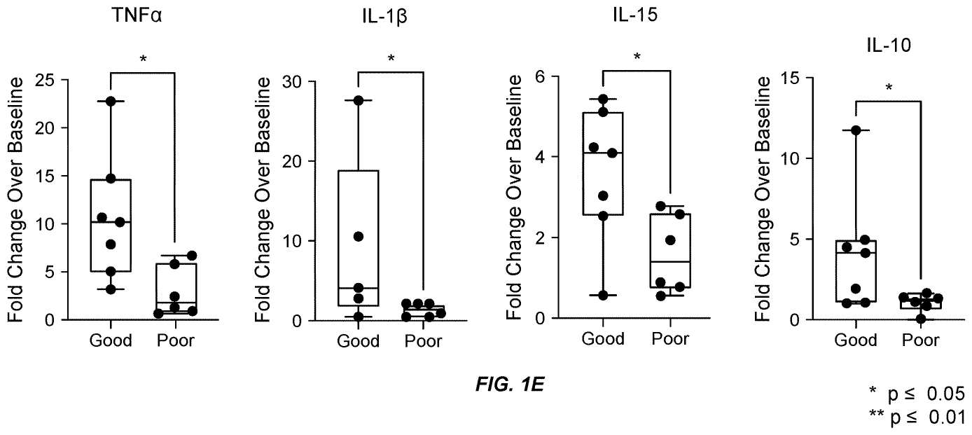

This is a Phase 1 dose escalation trial of GD2 CAR-Ts in children and young adults with GD2+ solid tumors. The trial used a 3+3 design with primary objectives to determine the feasibility of producing GD2 CAR-Ts meeting the established release criteria and to assess the safety of administering escalating doses of GD2 CAR-Ts in children and young adults with GD2+ solid tumors, including neuroblastoma and osteosarcoma, following cyclophosphamide-based lymphodepletion (FIG. 1A). Secondary objectives included determining if administration of GD2 CAR-Ts mediated antitumor effects, measure correlates of CAR-T cell activity, including CAR expansion and persistence. Patients were eligible for enrollment if they had a confirmed diagnosis of osteosarcoma or neuroblastoma, two tumors which have ubiquitous expression of GD2¹⁸.

### CAR-T Cell Manufacturing

Patients underwent apheresis to obtain autologous peripheral blood mononuclear cells (PBMCs). These cells were then selected using either bead selection or elutriation and ACK lysis, activated using CD3/CD28 beads, transduced with a bicistronic retroviral vector including an iCasp9 domain and a GD2.CD28.Ox40.z CAR (FIG. 1B), and then expanded for 7-9 days with IL-2²⁵.

### Cytokine Assay

Plasma was cryopreserved before measurement of cytokines in a multiplex format according to manufacturer's instructions (Mesoscale Discovery, Gaithersburg, MD, USA).

qPCR Assay

GD2 CAR-T expansion was measured by qPCR of peripheral blood isolated PBMCs using TAQMAN chemistry on the STEPONEPLUS™ Real-Time PCR System according to manufacturer's instructions. See SEQ ID NOs: 6-10.

### Mass Cytometry, Cytometry by Time of Flight (CyTOF), Assay and Analysis

Pre-treatment (apheresis), GD2 CAR-T product, and post-treatment samples were analyzed by mass cytometry (CyTOF) with 2 different panels as previously described^{26,27}.

### CyTOF Assays

Upon thawing, cells were washed twice with RPMI supplemented with penicillin/streptomycin and L-glutamine (Hyclone), 10% FBS (Hyclone), 20 U/mL of sodium heparin (Millipore-Sigma) and benzonase 25 U/mL (Pierce, ThermoFischer). Cell counts were obtained using a Vi-Cell XR cell viability analyzer (Beckman Coulter), and cells were split to proceed with antibody staining with a range of 1 to 2 million cells per test. Reconstituted lyophilized Veri-Cells tagged with 181Ta (custom order from Biolegend) were added directly to each sample to a ratio 1:10.

For the T cell Phenotype Panel, cells were washed twice in PBS (Rockland) and then were stained for live-dead discrimination with Cell-ID™ Cisplatin 5 μM (Fluidigm) for 5 min. After 2 washes with the Cell Staining Solution (CSM, PBS 1× supplemented with 0.5% BSA and 0.02% sodium azide), cells were resuspended in 1× MAXPAR® Fix I Buffer (Fluidigm) and fixed for 10 min at room temperature. Cells were washed twice with MAXPAR 10× Barcode Perm Buffer (Fluidigm) and stained for barcoding with the Cell-ID™ 20-Plex Pd Barcoding Kit (Fluidigm) for 30 min at room temperature. Then, cells were washed twice with CSM prior to be pooled in a unique 5 mL FACS tube. The composite sample was stained with surface antibody cocktail (Supplementary Table X) containing the Fc receptor blocking solution HUMAN TRUSTAIN FCX™ (Biolegend) for 30 min at room temperature. After 2 washed with CSM, cells were fixed in CSM+PFA 2% (AlfaAesar) for 10 min at room temperature and wash again in CSM. Then cells were fixed again with ice-cold methanol for 10 min on ice in the dark. After 2 washes with CSM, we proceeded to intracellular staining (Supplementary Table X) for 45 min at room temperature. Finally, after 2 washed in CSM, cells were stained with the CELL-ID IR-INTERCALATOR in 1×PBS (Rockland)+PFA 2% overnight at 4° C. Prior to acquisition on the next day, the sample was washed twice with CSM and 3 times with milliQ water and resuspended with EQ Four Element Calibration Beads (Fluidigm) 1:10 in milliQ water. Data was acquired on a Helios mass cytometer (Fluidigm).

### CyTOF Analysis

For the T cell Phenotype Panel, after CyTOF acquisition, the data collected were normalized using the Nolan Lab normalizer (github [dot] com/nolanlab/beadnormalization/releases). Samples from the T cell Phenotype Panel were deconvoluted with the Zunder Lab Single Cell Debarcoder (github [dot] com/zunderlab/single-cell-debarcoder).

For the Myeloid Panel, during data acquisition, a CD45-barcoding method was used to add healthy PBMCs as a spike-in technical control to the samples. After bead-based normalization using the Normalizer²⁸, only live and singlet cells were included in the analysis. Data were batch corrected with a quantile function constructed for the pooled distribution of each batch (a pair of sample and spikein control) using the CYDAR package²⁹.

Analyses of the T cell Phenotype Panel data and the Myeloid Panel data followed the same pipeline. Data were arcsinh transformed (cofactor=5) and The CATALYST package³⁰ was used to apply the FlowSOM method³¹ to cluster the cells and project the cells on the UMAP. Cell types were identified using lineage markers. Most clusters were present in each patient. Generalized Linear Mixed Model (GLMM), through lme4 package³², was used to identify significant differential cell population abundances. Correlations between cell type frequencies and GD2-CART expansion (GD2 CAR copy numbers per 100 ng of detected DNA) were performed using Spearman rank correlation tests. For the myeloid analysis, diffusion map pseudotime was performed for trajectory analysis using the destiny package³³.

In the analyses of both CyTOF panel data, a machine learning (ML) strategy was implemented to discover the genes that can effectively discriminate between different conditions. To accomplish this goal, the importance scores of the genes were determined by training a random forest (RF) model using the normalized gene expression from the same number of randomly selected cells from each condition. The CARET and RANDOMFOREST R packages were used to apply the machine learning analysis^{34,35}. This procedure was repeated for 30 iterations and the importance scores of the genes in each iteration were scaled to the 0-100 range for a better comparison. In classifying the groups, a higher score is regarded as having more classification power. The RPART and RPART.PLOT R packages were used to visualize a representative decision tree from the random forest model^{36,37}.

### RNA-Sequencing Assay and Analysis

cDNA Library Construction

Total RNA was quantified using the QUANT-IT™ RIBOGREEN® RNA Assay Kit and normalized to 5 ng/μl. An aliquot of 200 ng for each sample was transferred into library preparation which was an automated variant of the ILLUMINA TRUSEQ™ STRANDED mRNA SAMPLE PREPARATION KIT. This method preserves strand orientation of the RNA transcript. It uses oligo dT beads to select mRNA from the total RNA sample. It is followed by heat fragmentation and cDNA synthesis from the RNA template. The resultant 500 bp cDNA then goes through library preparation (end repair, base ‘A’ addition, adapter ligation, and enrichment) using Broad designed indexed adapters substituted in for multiplexing. After enrichment the libraries were quantified with qPCR using the KAPA LIBRARY QUANTIFICATION KIT FOR ILLUMINA SEQUENCING PLATFORMS and then pooled equimolarly. The entire process is in 96-well format and all pipetting is done by either Agilent Bravo or Hamilton Starlet.

### Illumina Sequencing

Pooled libraries were normalized to 2 nM and denatured using 0.1 N NaOH prior to sequencing. FLOWCELL cluster amplification and sequencing were performed according to the manufacturer's protocols using either the HISEQ 2000 or HISEQ 2500. Each run was a 101 bp paired-end with an eight-base index barcode read. Data was analyzed using the Broad Picard Pipeline which includes de-multiplexing and data aggregation.

### RNA-Seq Analysis

Paired-end transcriptome reads were processed with a standardized RNA-seq IMmune Analysis pipeline called RIMA (https://liulab-dfci.github.io/RIMA). RIMA is an automated Snakemake pipeline to streamline the processing of RNA-seq data. Read alignments were performed with STAR³⁸ against the hg38 reference genome from the NCI Genomic Data Commons (GDC). RNA-seq quality control was performed on the aligned BAM files using RSeQC³⁹. With the reads appropriately aligned, transcriptome per million (TPM) were quantified by SALMON⁴⁰ on the BAM files. Differentially expressed genes were identified using DESeq2⁴¹. Gene set enrichment was performed using CLUSTERPROFILE⁴² against HALLMARK and IMMUNESIGDB gene sets from the MOLECULAR SIGNATURE DATABASE (MSigDB)⁴³. Immune infiltration analysis was performed using CIBERSORT⁴⁴ from the IMMUNEDECONV⁴⁵ R package. Gene expression was first normalized by subtracting the average expression across samples, and the gene signature score was then calculated as the average normalized expression of gene sets. Wilcoxon rank-sum test was then applied to the gene signature scores.

### ATAC-Seq Assay and Analysis

Assay for Transposase-Accessible Chromatin with high-throughput sequencing (ATAC-seq) was conducted as previously described⁴⁶ with minor modifications specified below. Apheresis and Product samples were counted, and 150,000 cells were split into 3 replicates containing 50,000 cells each. Cells were pre-treated with 200 units/ml DNase I (Worthington Biochemical, LS006343) in PBS for 30 min at 37° C. Post-incubation, samples were washed twice with ATAC resuspension buffer (ATAC-RSB; containing 1M Tris-HCl pH 7.5, SM NaCl, 1M MgCl₂ in sterile water), plus 0.1% Tween-20, with centrifugation at 500 g for 5 minutes at 4° C. Cells were then lysed for 3 minutes on ice with 50 μl cold ATAC-RSB plus 0.1% NP40, 0.1% Tween-20 and 0.01% Digitonin. After lysis, nuclei were washed with 1 ml ATAC-RSB containing 0.1% Tween-20 and were pelleted at 500 g for 10 minutes at 4° C. Nuclei were transposed with Illumina Tn5 transposase (Illumina, 20034198), and reactions were cleaned up with the QIAGEN MINELUTE PCR PURIFICATION KIT (Qiagen 28006). ATAC libraries were PCR amplified with NEW ENGLAND BIOSYSTEMS' 1× PCR MASTER MIX (NEB, M0541L) and custom Nextera PCR primers at 1.25 μM each for 11 or 12 total cycles. Amplified libraries were purified with AMPURE XP beads (Beckman Coulter A63880), and the KAPA library quantification kit (Kapa Biosystems, KK4854) was used to quantify library concentrations. Libraries were pooled with equimolar concentrations and sequenced by Novogene on a NOVASEQ S4 with 2×150 bp paired-end reads.

### Results

### Patient Characteristics and CAR T Cell Manufacturing Feasibility

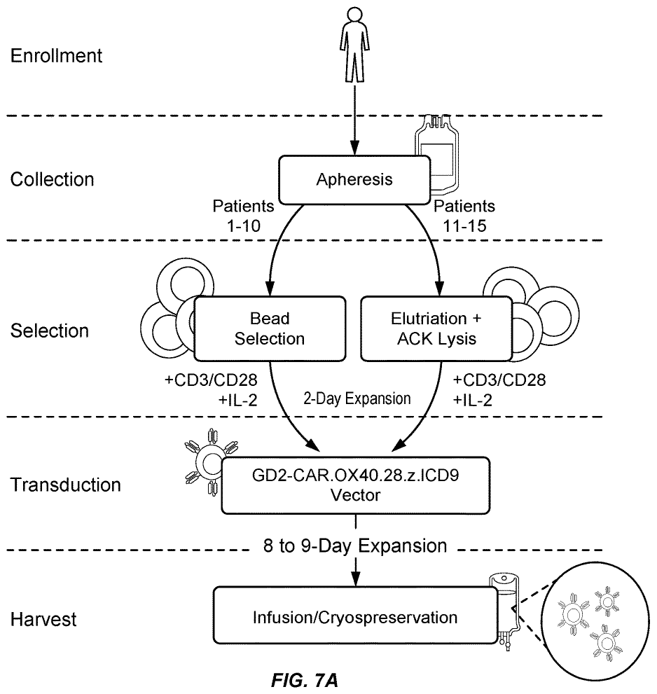

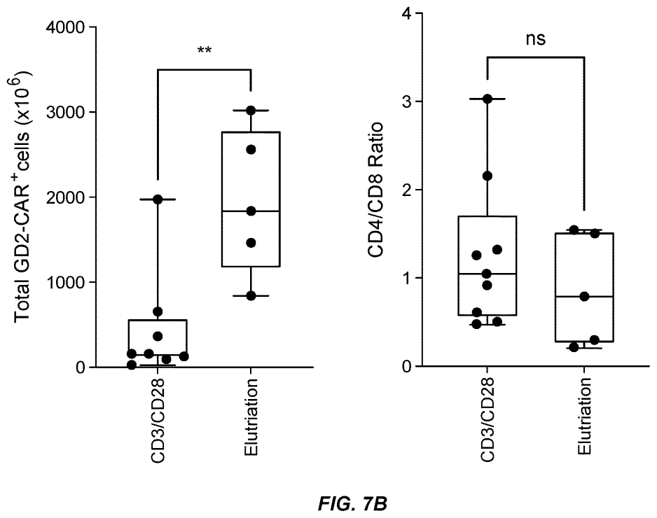

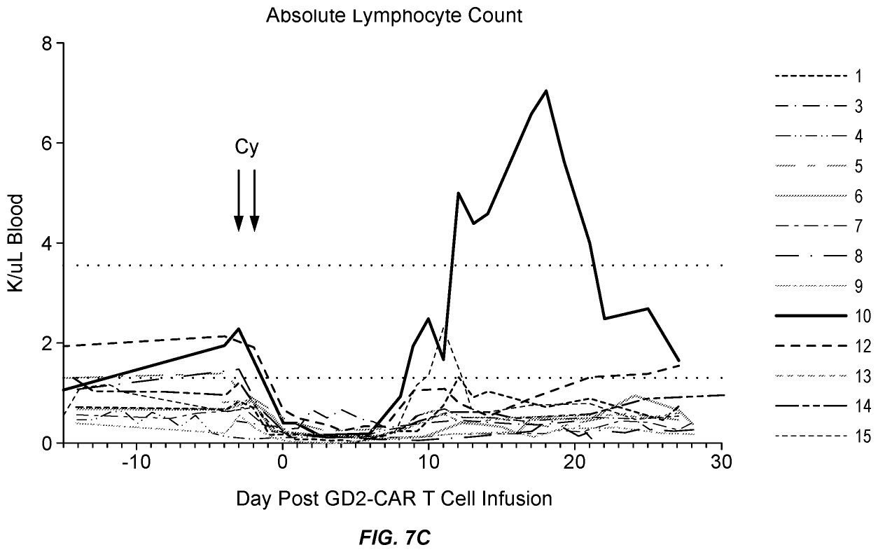

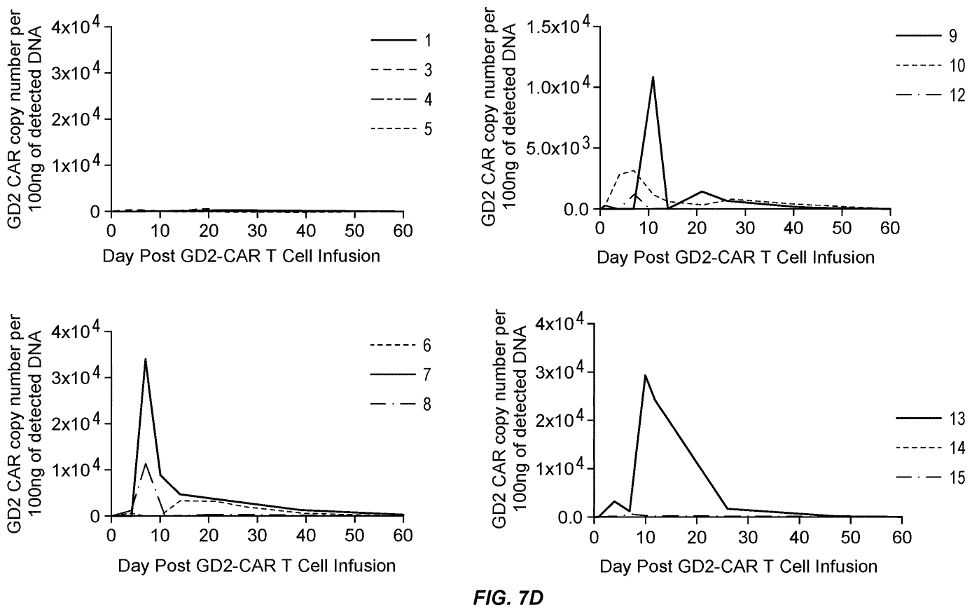

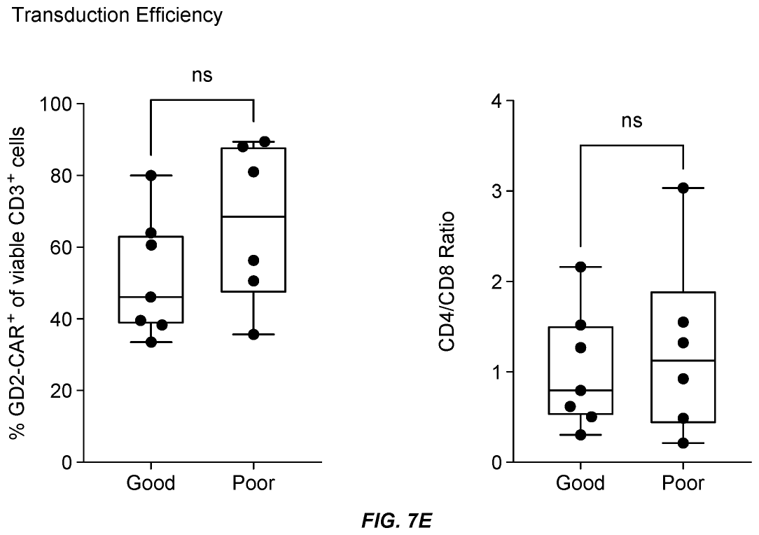

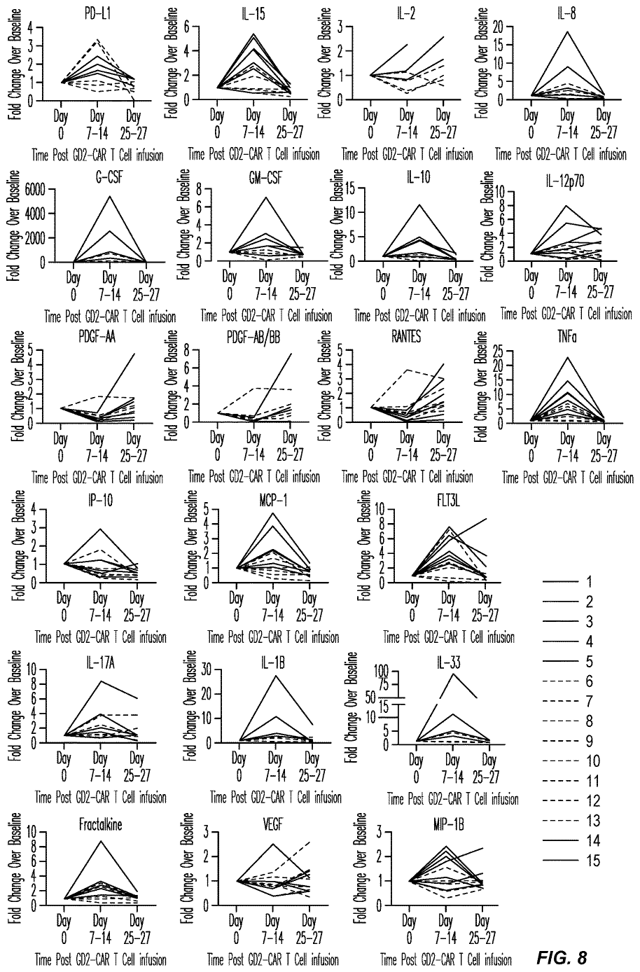

Fifteen patients were enrolled on this Phase 1 clinical trial (NCT02107963) testing GD2.CD28.Ox40.z CAR-Ts in patients with osteosarcoma or neuroblastoma (Table 1, FIG. 1A-B). The median age was 17 (range 8-28), with 12 patients with osteosarcoma and 3 with neuroblastoma. These patients were all heavily pretreated with multiple modalities of therapies (Table 1). Thirteen patients received the intended GD2 CAR-Ts, two patients died prior to CAR-T administration. GD2 CAR-Ts were manufactured using a retroviral vector (IC9-2A14G2A.CD28.OX40Z), activated by CD3/CD28 beads and IL-2, with manufacturing details represented (FIG. 1B; FIG. 7A). The selection process prior to manufacturing changed from bead selection to elutriation+ACK Lysis for the last four patients to reduce myeloid populations during the manufacturing process²⁵ and resulted in improved GD2 CAR-T expansion (FIG. 7B). One patient failed transduction/expansion based on the final number of GD2 CAR-T transduced cells (6e⁶ cells at harvest) but was able to be re-manufactured with the addition of ficoll density gradient and monocyte adhesion steps. All GD2 CAR-T products were manufactured within 10-11 days and met release criteria as specified on the clinical trial protocol.

### Toxicity and Response

Overall, GD2 CAR-Ts were very well tolerated without significant evidence of toxicity (Table 3). 15.4% (2/13) of patients experienced grade1 cytokine release syndrome (CRS), and no neurological toxicity was observed. No dose-limiting toxicities were observed in any of the dose levels of administration. At Day 28 following GD2 CAR-T infusion, 23.1% (3/13) of evaluable patients had progressive disease and 76.9% (10/13) had stable disease (SD). 3/10 SD patients remained stable at 60 days post-GD2 CAR-T, but all patients eventually progressed (Table 2).

### CAR-T Cell Kinetics and Activity

While subsequent CAR-T trials have implemented fludarabine and cyclophosphamide for lymphodepletion, this trial used only cyclophosphamide, resulting in a nadir in absolute lymphocyte count occurring between day 0 and 7 in all patients (FIG. 7C). We measured the expansion and persistence of adoptively transferred GD2 CAR-Ts in the peripheral blood using qPCR. GD2 CAR-Ts expanded in all patients receiving treatment, half of whom had expansion above 1000 GD2 CAR copies per 100 ng DNA, a level similar to that seen in clinically active CD19 and CD22 CAR-Ts. However, the GD2 CAR-Ts had limited persistence (FIG. 1C; FIG. 7D). CAR-T expansion did not associate with dose level, CD4/8 ratio, or CAR transduction efficiency in the CAR-T product (FIGS. 7E-F). Interestingly, peak GD2 CAR-T expansion did associate with increased pro-inflammatory cytokines in patients, suggesting a functional difference in these patient CAR-Ts (FIG. 1E; FIG. 8)47. Additionally, all patients with good CAR-T expansion demonstrated stable disease, whereas of poor responders, 3/5 patients had progressive disease within the 28-day window.

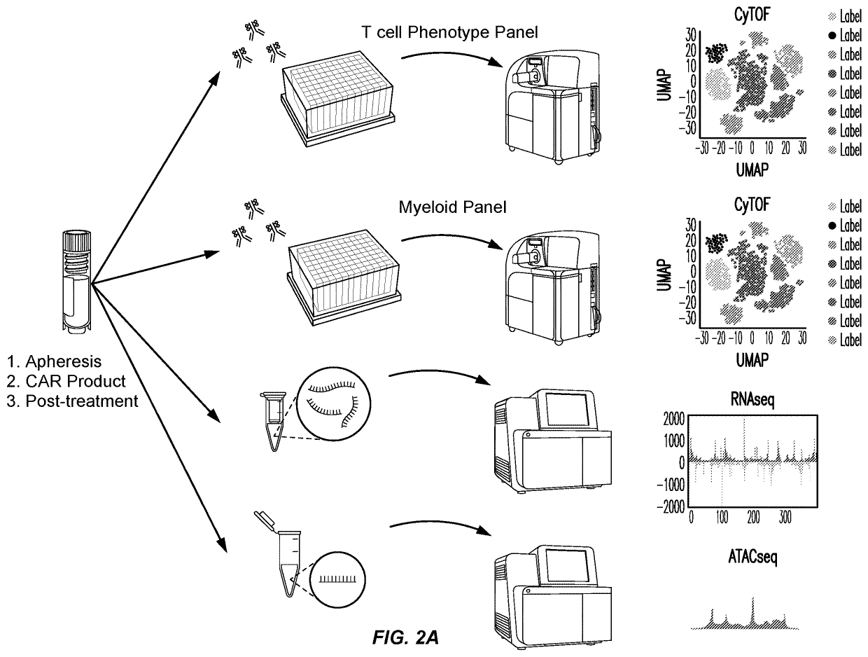

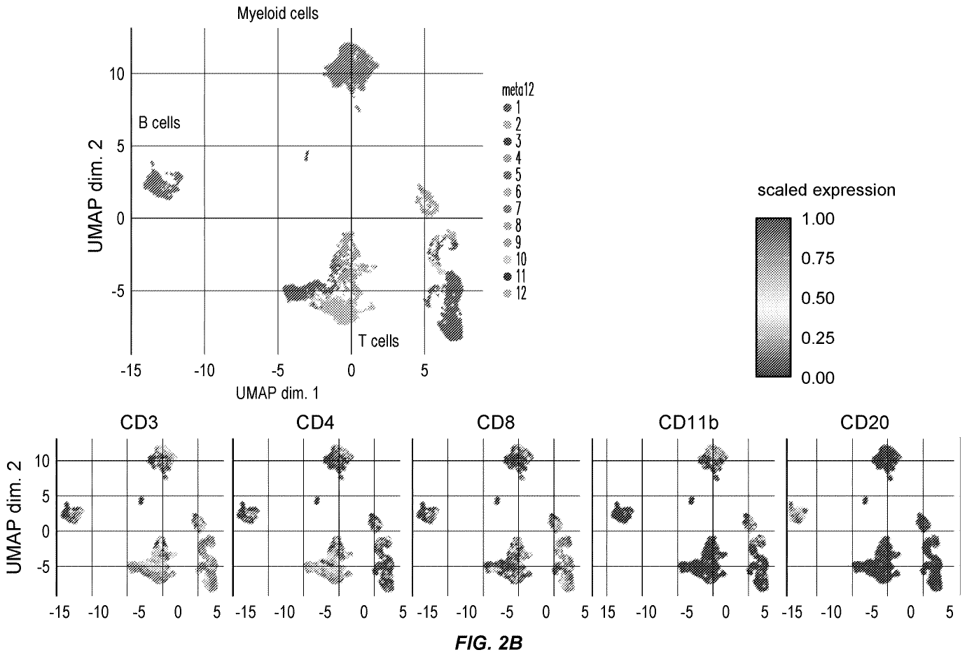

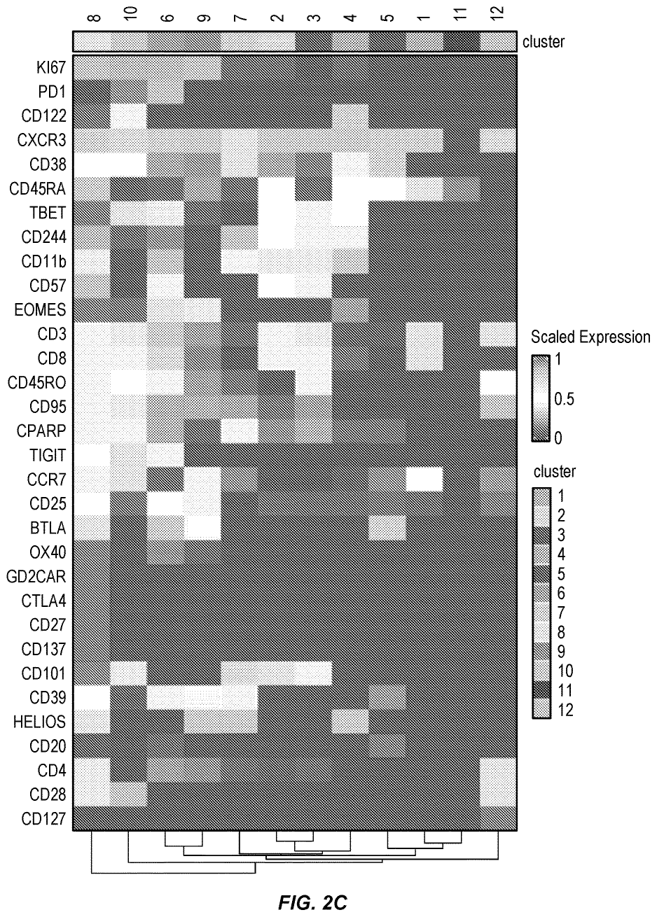

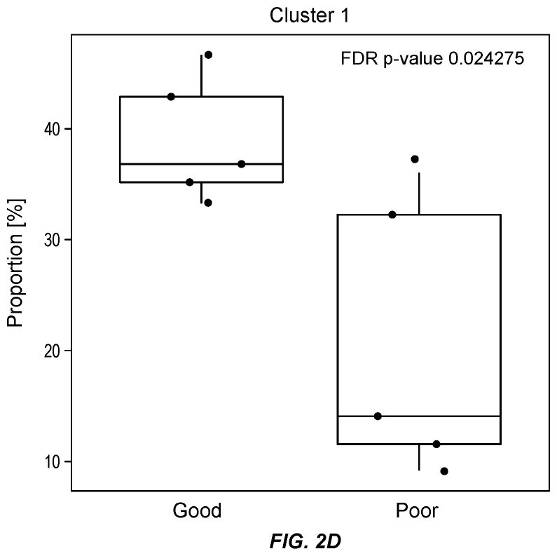

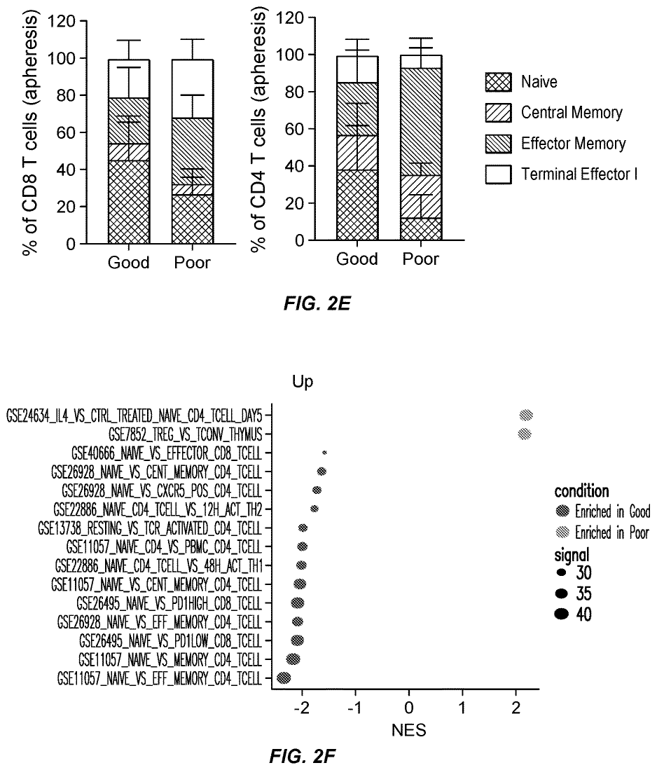

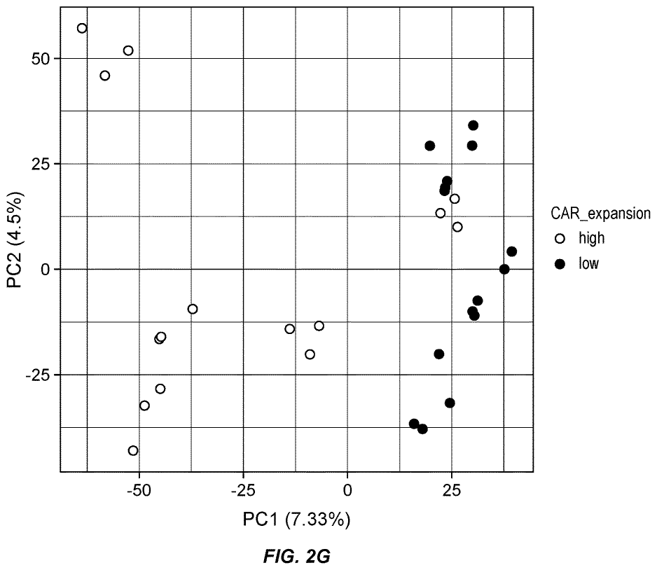

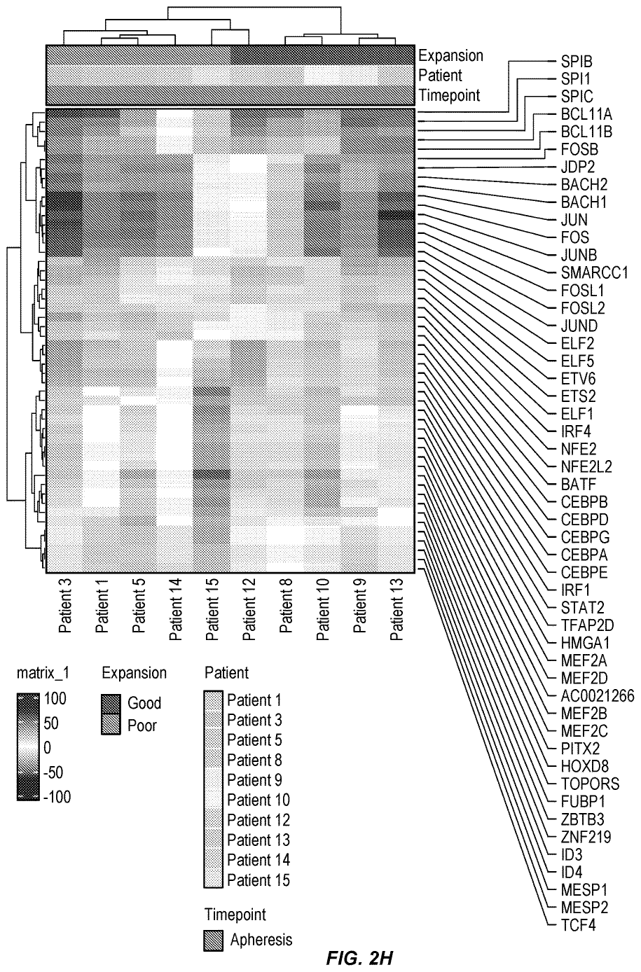

To understand the cellular correlates of CAR-T expansion and activity in these patients, we performed comprehensive phenotypic (CyTOF), transcriptomic (RNAseq), and epigenetic (ATAC-seq) analyses of patient samples at pre-treatment apheresis (apheresis), CAR-T cell product (product), and post-CAR-T infusion (post-treatment) timepoints (FIG. 2A).

Good CAR-T Cell Expansion Associated with Improved T Cell Memory Subsets Prior to CAR-T Cell Manufacturing

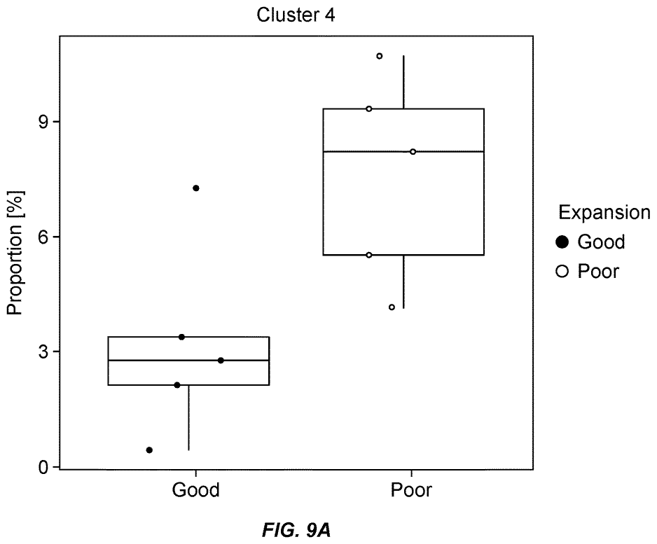

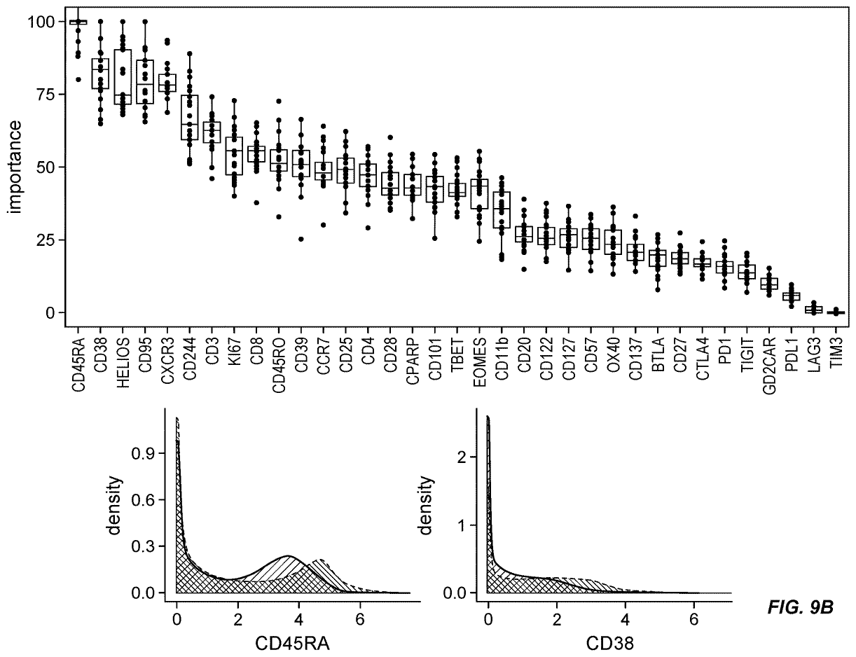

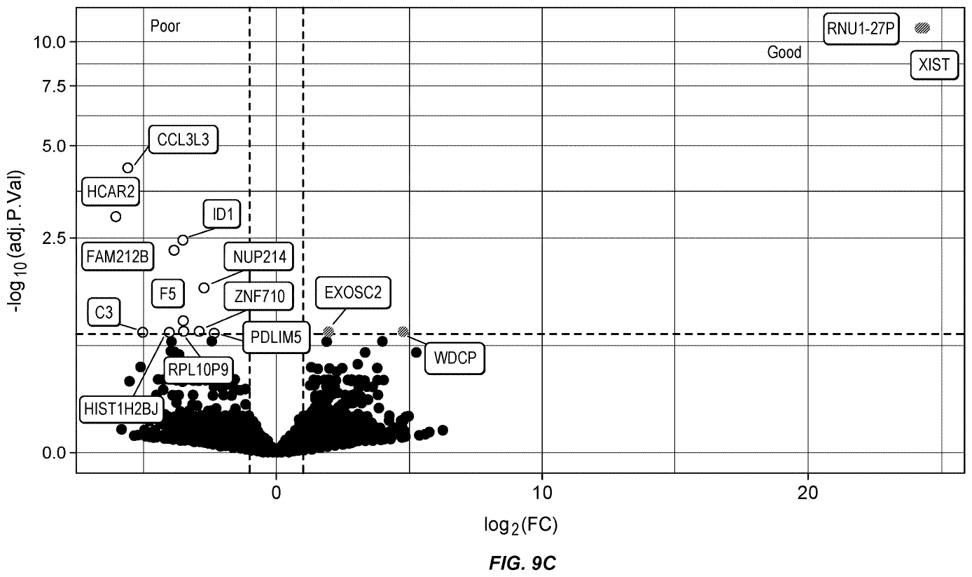

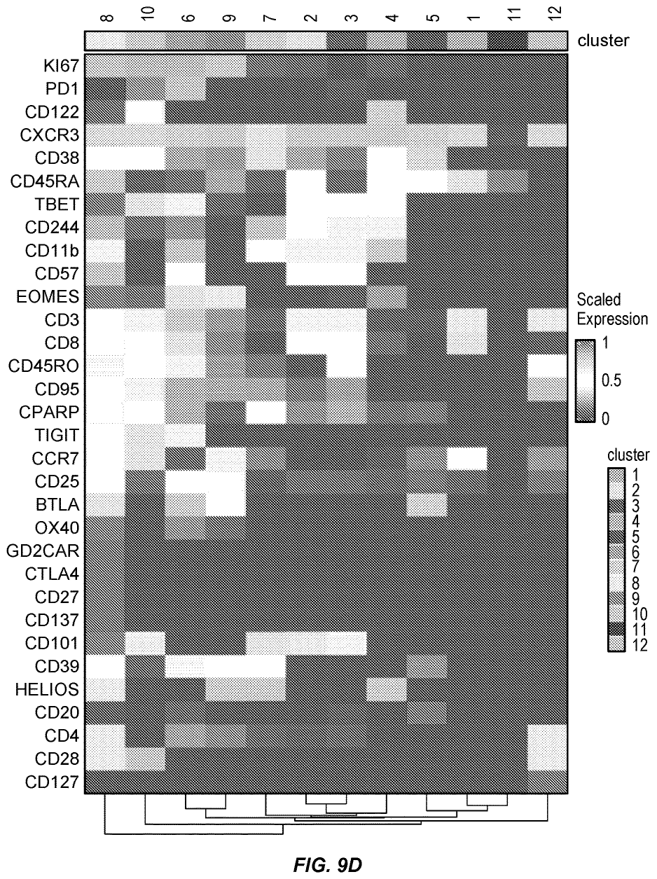

Analysis of T cell phenotype of patient apheresis samples by CyTOF using the T cell Phenotype Panel identified 12 distinct clusters (FIG. 2B-C). Comparison of samples originating from good versus poor expanders identified cluster 1, a naïve memory CD8 T cell population associated with good CAR-T cell expansion, (FIG. 2D: CD3+CD8+CD45RA+CCR7+; FDR-adjusted p-value 0.024275), and cluster 4, a terminal effector TEMRA CD8 T cell population associated with poor CAR-T cell expansion (FIG. 9A: CD3+CD8+CD11b+CD122+CD38+TBET+CD45RA+CCR7−; FDR-adjusted p value of 0.054426). Manual gating of apheresis memory populations based on CD45RA and CCR7 confirmed that naïve (CD45RA+CCR7+) CD4+ and CD8+ T cells trend to increased levels in good expanders, while terminal effector TEMRA (CD45RA+CCR7−) CD4+ and CD8+ T cell populations were predominant in poor expanders (FIG. 2E). Random forest analysis of apheresis samples identified CD38, previously implicated in antigen-induced T effector function48, as an important marker associated with poor expansion (FIG. 9B). Although RNA-seq analysis identified limited differentially expressed genes between good and poor expanders (Figure C), GSEA pathway analysis focusing on T cell pathways identified enrichment of pathways associated with naïve or central memory phenotypes in good expanders' apheresis (FIG. 2F). Similarly, epigenetic analysis of apheresis samples via ATAC-seq identified PCA separation between good and poor expander apheresis samples (FIG. 2G). TF motifs such as FOS, JUNB, and JUND were reduced in good expanders (FIG. 2H). These findings illustrate that naïve memory T cell populations in apheresis correlate with good CAR-T expansion, whereas TEMRA T cell populations in apheresis associate with poor expansion.

### Good and Poor Expanders Exhibited Shared Immune Cell Populations in Product and Post-Treatment Samples

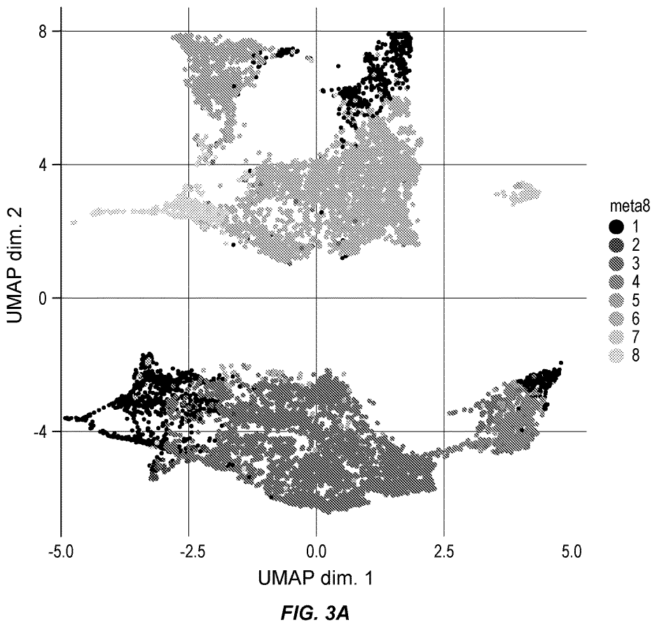

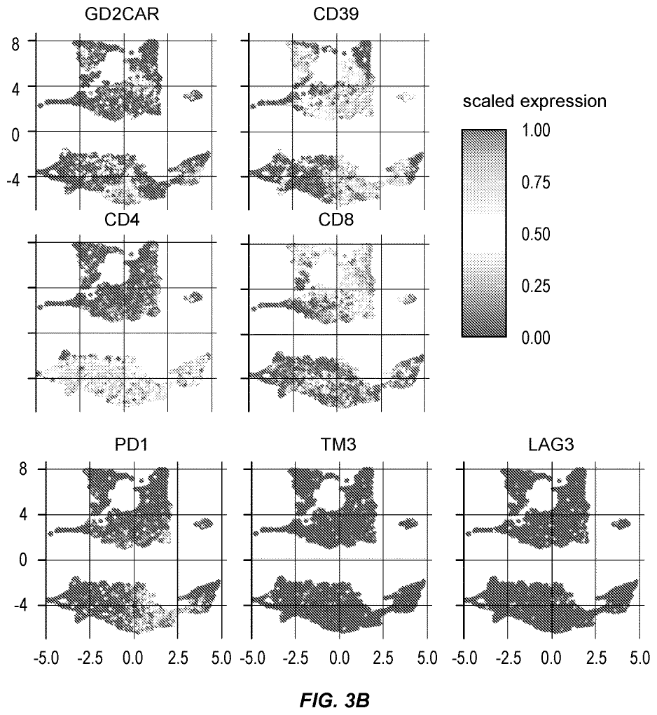

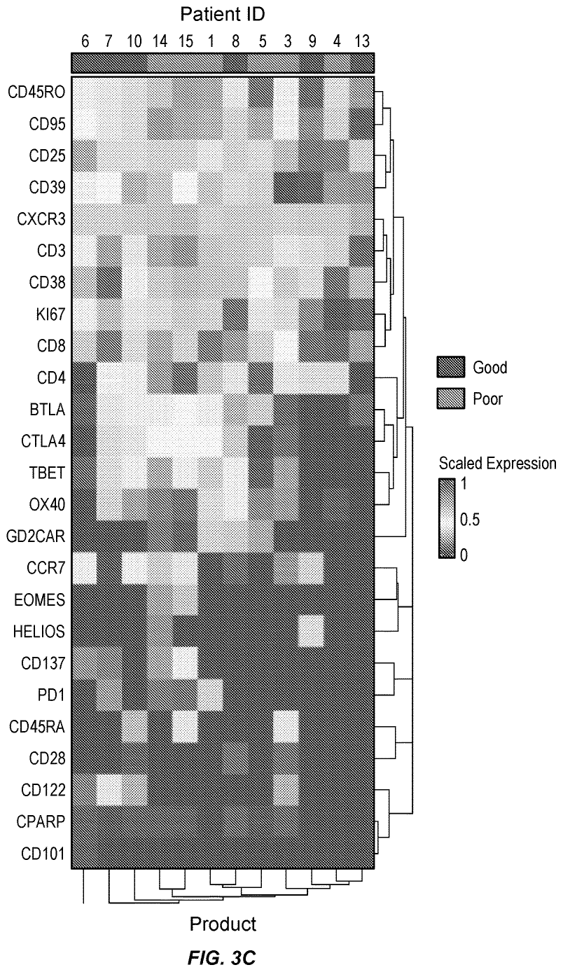

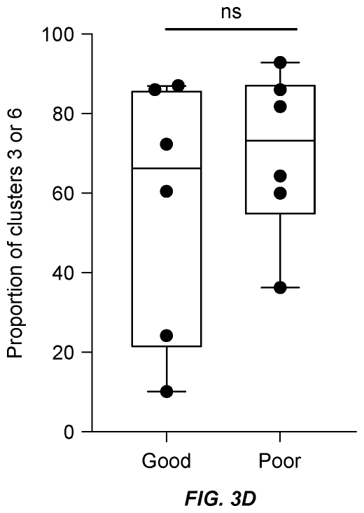

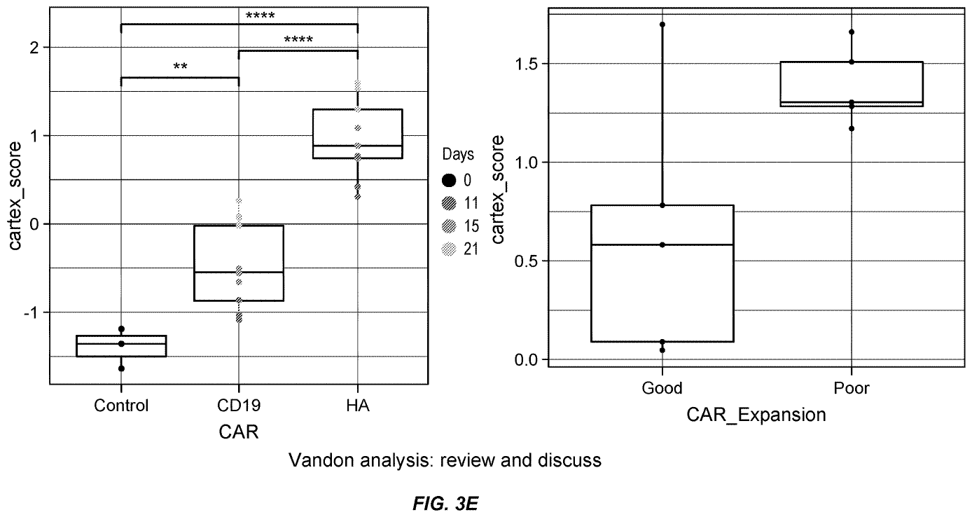

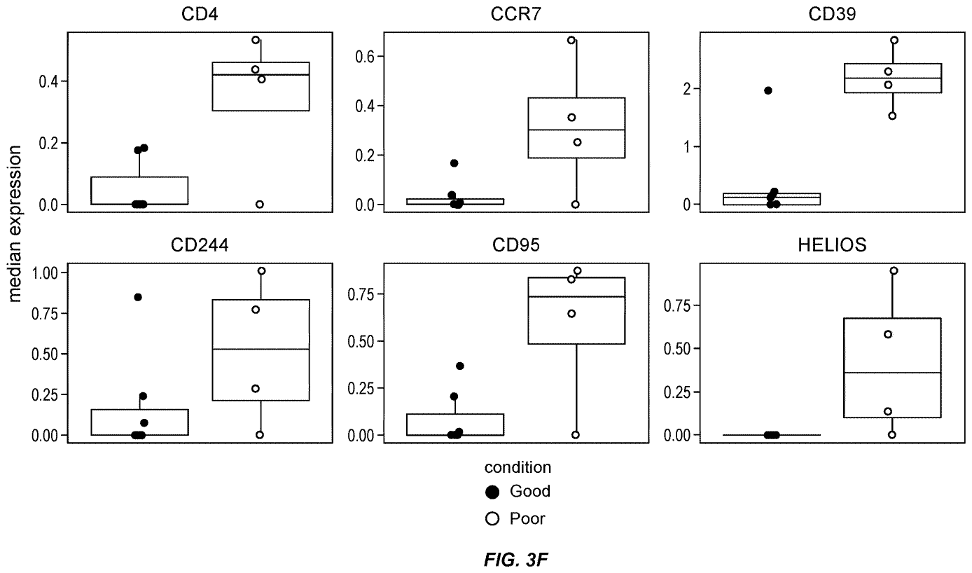

T cell Phenotype Panel CyTOF analysis of GD2 CAR-T products identified 8 distinct clusters but did not identify any differences in cluster abundance between good and poor expanders (FIG. 3A-C; FIG. 9D). Interestingly, clusters 3 and 6 comprise an average of 63% of each patient's product (standard error 7.8) (FIG. 3D). These GD2 CAR+ T cell clusters include a proliferating activated CD4+GD2 CAR-T cluster (Cluster 3; CD45RO+OX40+CD38+TBET+CTLA4+BTLA+CD39+KI67+CD95+) and a proliferating activated CD8+ GD2 CAR-T cluster (Cluster 6; D45RO+CD38+TBET+CTLA4+BTLA+CD39+KI67+CD95+) (FIG. 9C). RNAseq analysis of a CAR-T exhaustion score (CARTEx score), which highlights exhaustion signatures in previously published data49, identified that the CARTEx score was increased in poor CAR-T expander products (FIG. 3E). These data suggest that while CAR-T products were similar across all patients, poor expander samples demonstrated transcriptomic differences suggesting increased transcriptional exhaustion programing. When evaluating post-treatment samples, these poor expanders demonstrated increased CD39 expression, consistent with an exhaustion phenotype50,51 (FIG. 3E). Together, these finding suggest that CAR-T products with transcriptomic increased CARTEx scores result in more exhausted T cells following CAR-T administration with correlating decreased expansion and persistence.

Myeloid Cell Populations are Associated with CAR Expansion

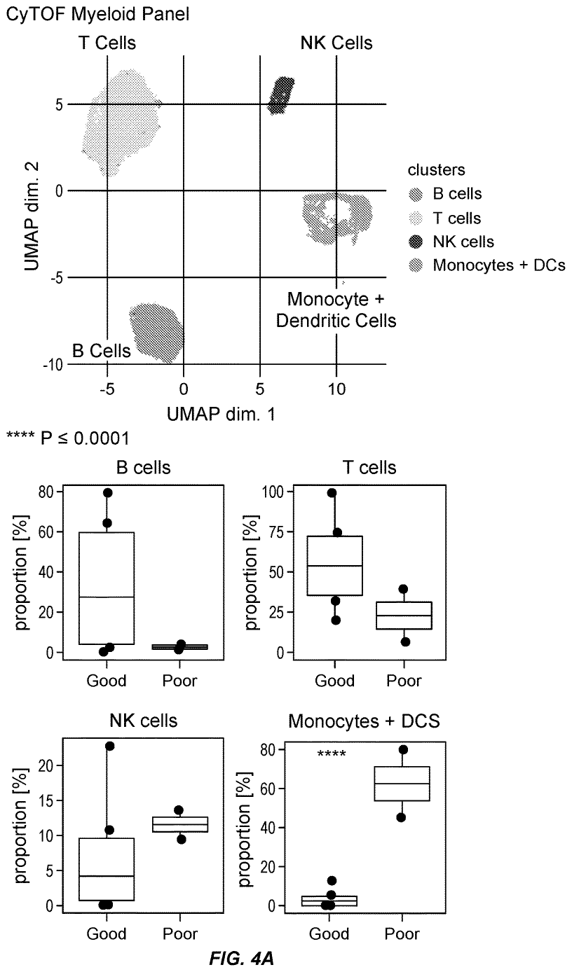

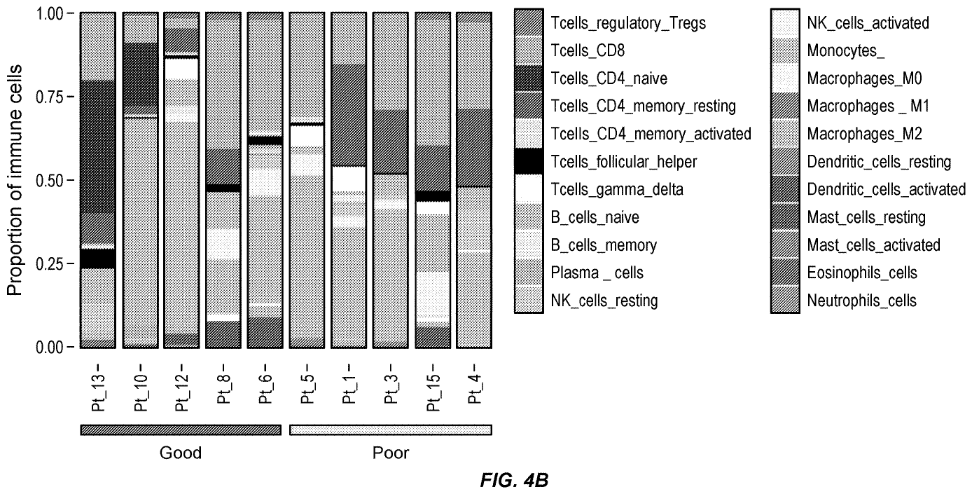

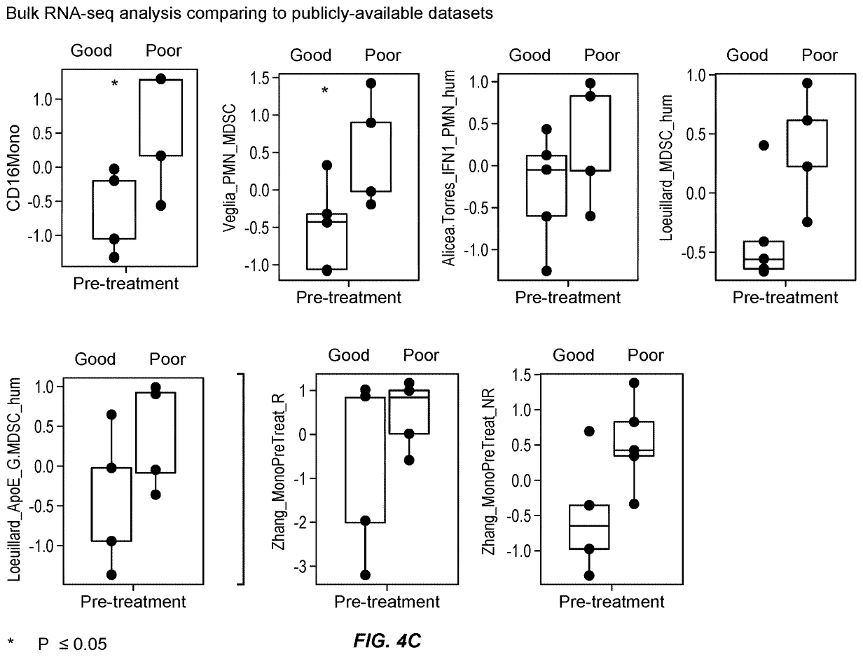

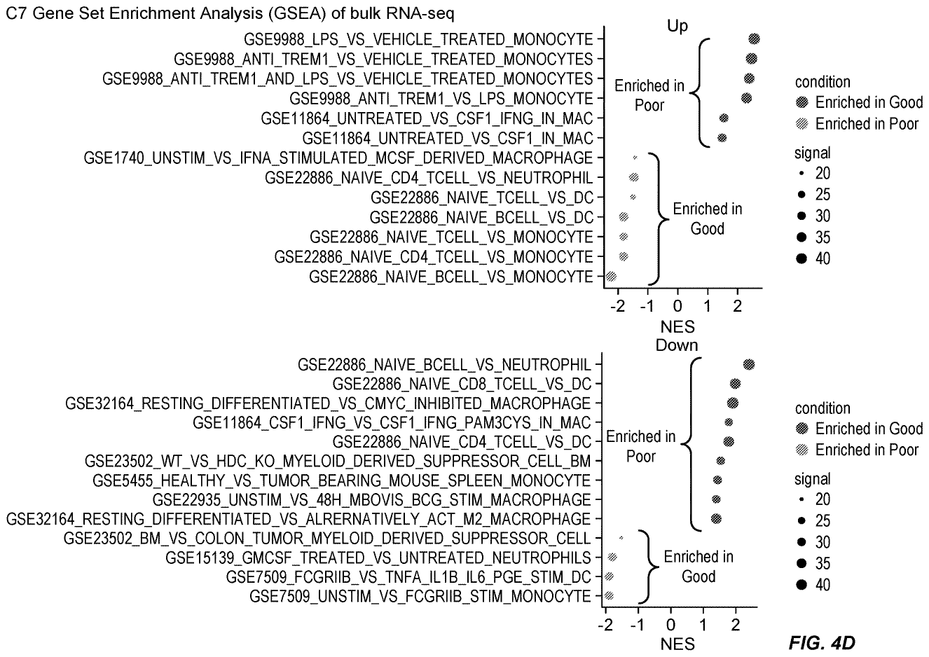

Given the crucial role that myeloid cells play in orchestrating immune responses and a growing body of literature demonstrating the immunosuppressive role that myeloid cells can play in limiting immune responses in solid tumors, we sought to characterize the myeloid populations in patients receiving GD2-CAR therapy. Clustering analysis of CyTOF data demonstrated significantly reduced myeloid cells, namely monocytes and dendritic cells (DCs), in patients with good versus poor CAR expansion (FIG. 4A). CIBERSORT RNA sequencing analysis corroborated these findings that patients who experienced poor CAR expansion had a greater proportion of monocytes in their baseline apheresis (FIG. 4B). Using previously published datasets characterizing myeloid populations in the setting of cancer, we found that poor responders had increases in signatures associated with CD16 monocytes⁵² and myeloid derived suppressor cells (MDSCs)^{53,54} (FIG. 4D). Further, patients with poor CAR expansion had gene signatures resembling nonresponders to PD-L1 inhibition⁵⁵, suggesting a phenotype that limits other immunotherapeutic approaches in addition to CAR-T cells (FIG. 4D). GSEA analysis indicated that samples from patients with poor CAR expansion were enriched in pathways associated with an inflammatory response, such as LPS and TREM1 mediated pathways⁵⁶, in parallel to reduced T and B cell signatures relative to myeloid populations (FIG. 4E). Finally, ATAC-seq epigenetic assessment identified myeloid drivers such as SPI1 and Est2^{57,58} increased in poor expanders (FIG. 2H).

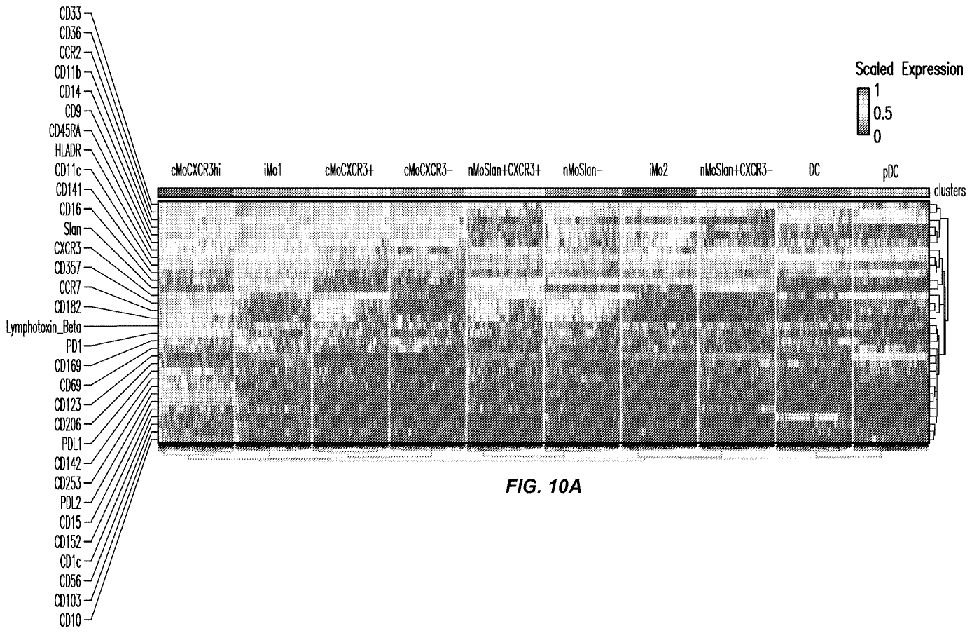

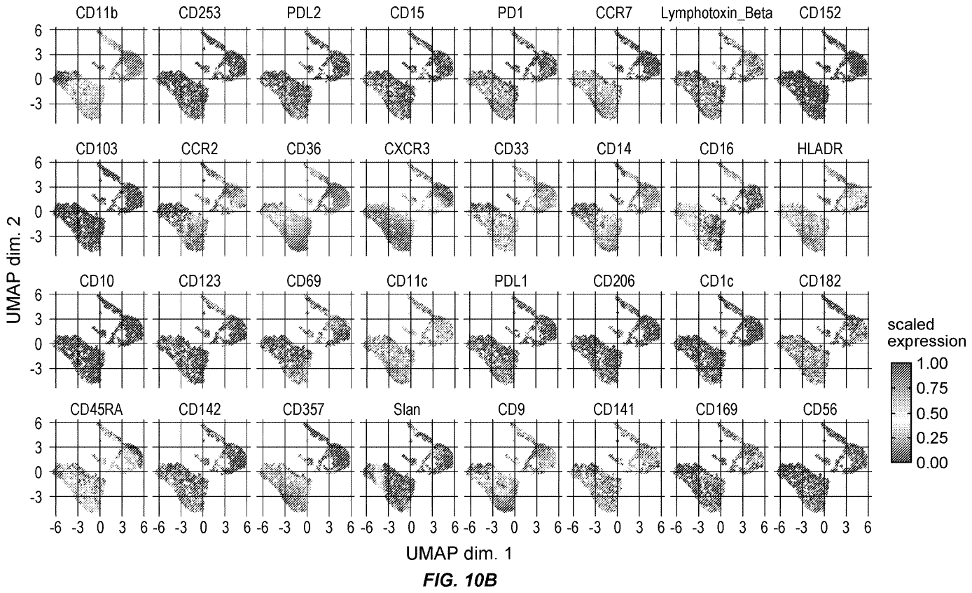

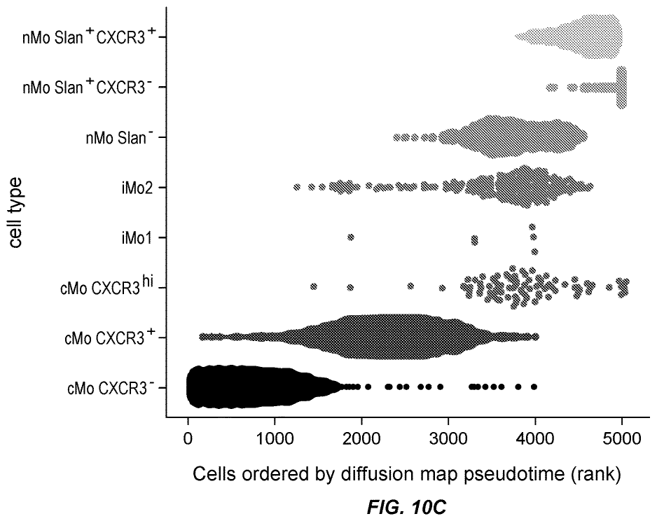

To provide more granularity to the differences in myeloid populations, we performed a sub-clustering analysis of the Myeloid Panel CyTOF data to focus on the monocyte and DC populations (FIG. 5A; FIGS. 10A-10B). CXCR3− classical monocytes and both clusters of intermediate monocytes were significantly increased in poor expanders, while CXCR3+ and CXCR3hi classical monocytes, as well as Slan− and Slan+CXCR3+ non-classical monocytes were significantly increased in good expanders (FIG. 5A). The multidimensional scaling (MDS) of these samples demonstrated that patients with poor expansion cluster together while good expanders have more diversity, indicating a commonality in the apheresis of poor expanders (FIG. 5B). In agreement with the prior T cell Phenotype Panel, the Myeloid Panel identified that patients with good CAR T cell expansion had increased expression of CCR7, CD45RA, GITR/CD357, CD19 and CD3 markers prior to CAR-T manufacturing (FIG. 5C). Random forest analysis shows that CXCR3 is the most robust marker of good CAR expansion. In contrast, patients who experienced poor CAR T cell expansion had increased expression of canonical monocyte markers CD11b and CD14, CD33 which is often used to identify MDSCs, in addition to the fatty acid transporter CD36, in their myeloid cell populations, mirroring pathways observed in the transcriptomic analyses (FIG. 5D)⁵²⁻⁵⁶.

Post-Treatment Patient Samples Suggest Myeloid Molecular Signatures Associated with CAR-T Cell Expansion

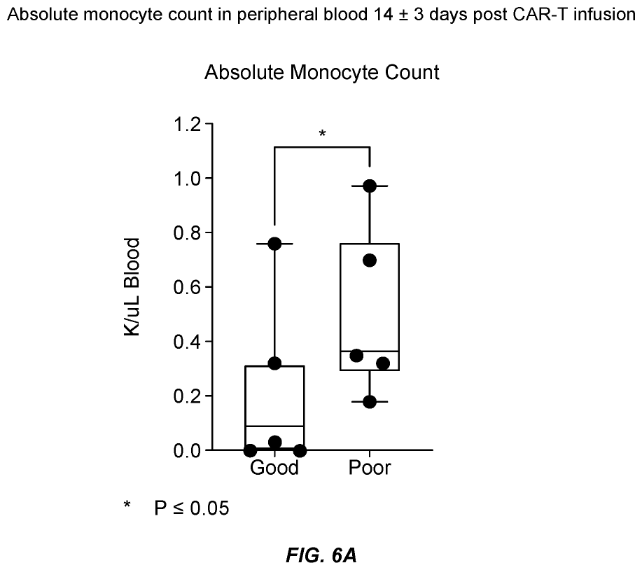

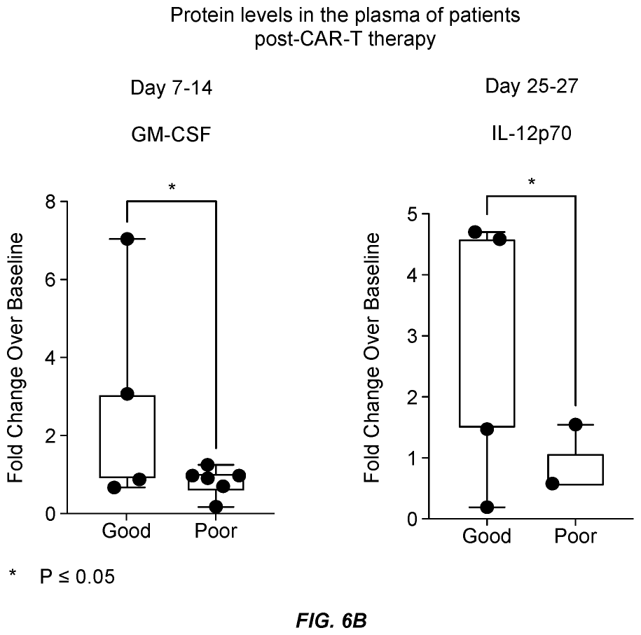

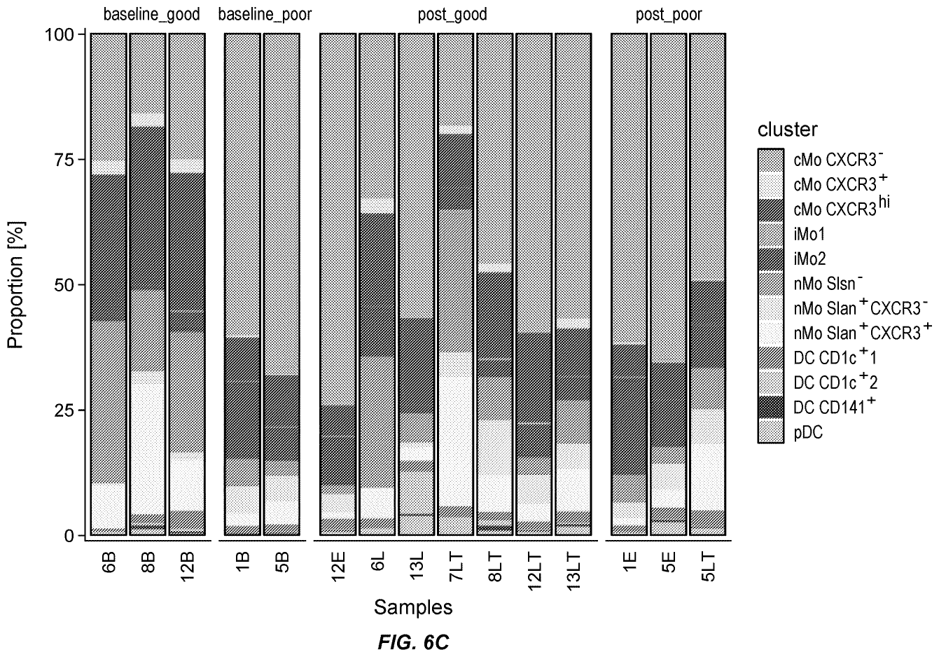

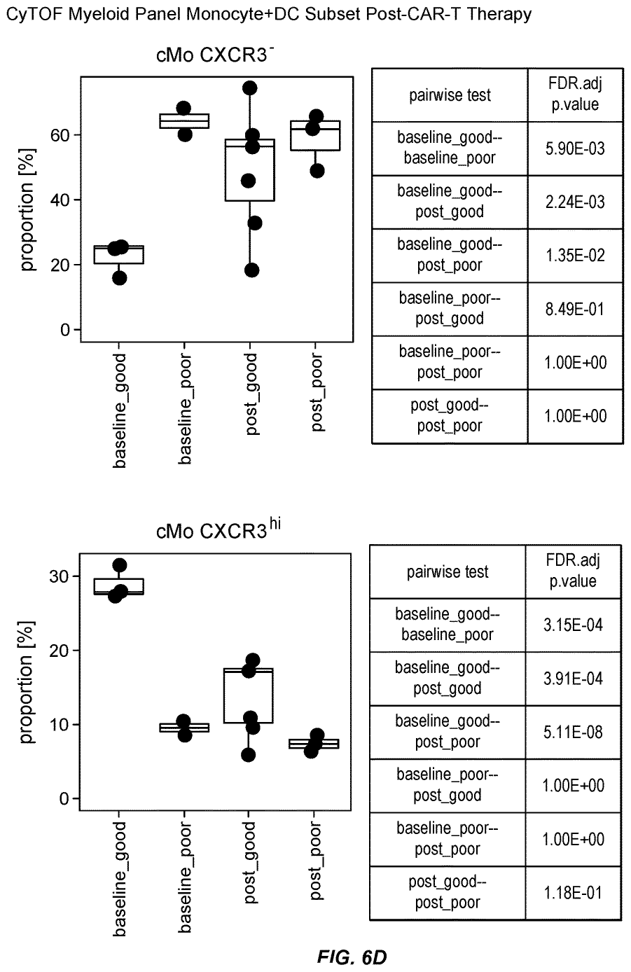

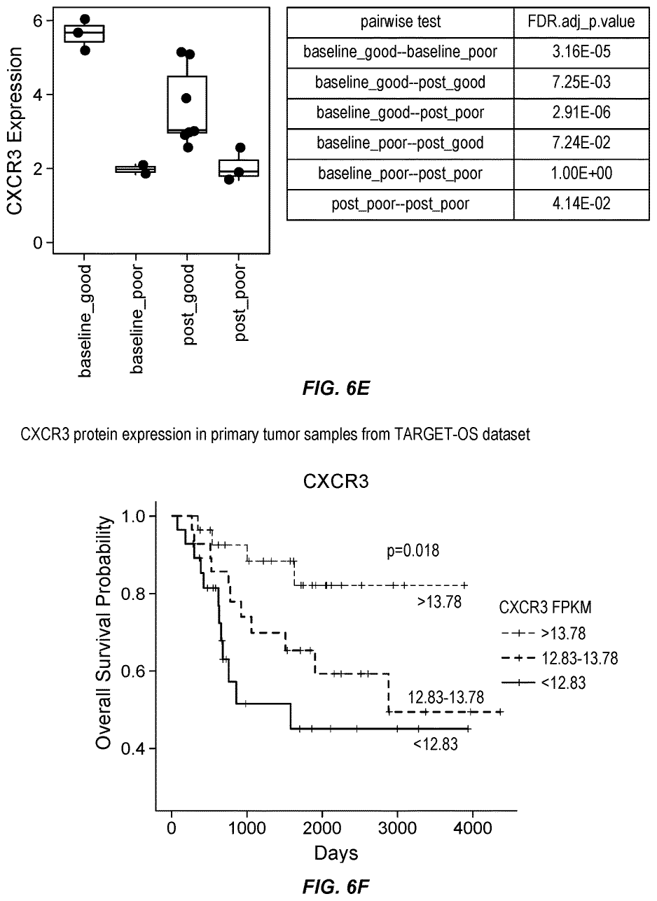

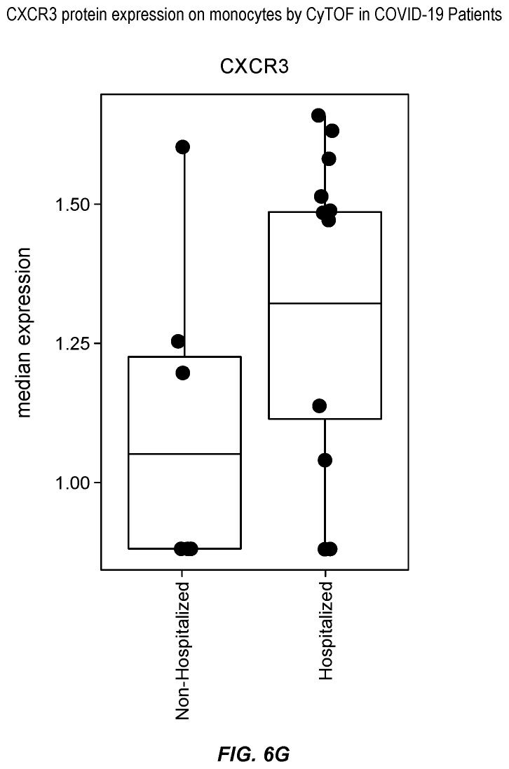

Beyond baseline differences of myeloid populations that could contribute to CAR T cell expansion, we next interrogated whether myeloid populations changed in response to CAR-T administration. Complete blood count (CBC) analysis showed that the absolute monocyte count (AMC) was significantly increased in poor CART expanders two weeks after treatment relative to those patients with good CART expansion (FIG. 6A), consistent with previous reports11. Cytokine analysis indicated that good expanders had increased GM-CSF and IL-12 in their plasma, which are associated with monocyte differentiation and antitumor responses, respectively (FIG. 6B). Myeloid Panel CyTOF analysis of peripheral blood immune populations showed significant increases in cMo CXCR3− and iMo2 clusters, with a significant decrease in the cMo CXCR3 hi cluster, following CAR-T cell infusion (FIG. 6C-D). Of note, poor expanders did not have differences in their monocyte populations pre- versus post-treatment, while the good expanders had monocyte populations that shifted from a favorable pre-treatment phenotype to a phenotype that resembles the poor expanders following CAR-T cell treatment (FIG. 6D). In fact, this shift maintained when evaluating CXCR3 expression on all myeloid cells (6E). These data suggest that the CXCR3− monocyte population associated with limited initial CAR-T expansion may also be responsible for limited CAR-T persistence in patients who experienced good CAR-T expansion. Trajectory analysis of the monocyte populations shows a progression from CXCR3-classical monocytes to Slan+CXCR3+ non-classical monocytes (FIG. 10C).

As CXCR3 expression was the most significant indicator of CAR-T expansion in our patients, we queried the TARGET-OS dataset and found a significant difference in survival, with high CXCR3 expression associated with significantly increased survival in patients with osteosarcoma and low CXCR3 expression associated with reduced survival (FIG. 6F). Additionally, we identified higher CXCR3 expression in hospitalized COVID-19 patients as compared to those not hospitalized59, suggesting increased immune activation in these patients (FIG. 6G). Together, these data demonstrate that monocyte populations, specifically CXCR3+ or CXCR3 hi classical monocytes, are associated with good CAR T cell expansion and provide evidence for a CAR T cell extrinsic, monocyte dependent mechanism contributing to CAR-T efficacy.

### Conclusion

The data discussed above in this Example demonstrate that CXCR3 expression on peripheral monocytes at baseline is associated with improved CAR-T cell expansion in vivo. Accordingly, this Example supports a conclusion that CXCR3 expression on myeloid cells is a predictive marker of immunotherapy efficacy and may be a useful target to improve immunotherapy. Thus, high CXCR3 expression on myeloid cells in pre-treatment apheresis product can be used as a predictive biomarker of effective immunotherapy.

### Example 2

This Example presents the results of experiments designed to further investigate the mechanism of CXCR3 on myeloid cells.

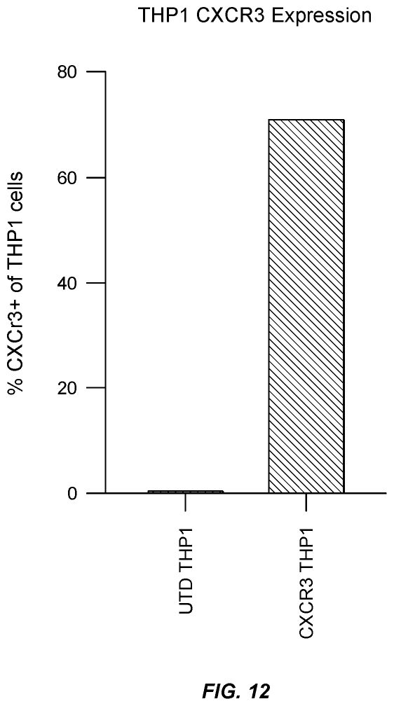

A CXCR3-overexpressing THP-1 cell line (CXCR3-THP1) was generated by lentiviral transduction. Briefly, a plasmid containing human CXCR3-A (NCBI Reference Sequence: NM_001504.2) was synthesized, and lentivirus was produced as previously described (Kaczanowska and Beury et al Cell 2021). THP1 cells were centrifuged in the presence of lentivirus and 8 mg/mL polybrene at 931×g for 2 hours at 30° C. Lentivirus was washed off the following day, CXCR3 expression was confirmed by flow cytometry (see FIG. 12), and stability of CXCR3 expression was maintained over time in culture and with freeze-thaw cycles.

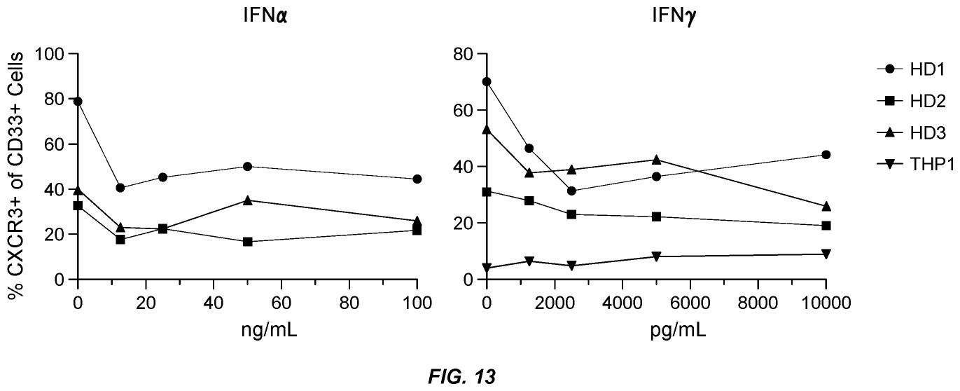

As CXCR3 expression can be induced by interferon signaling, and since immune cells produce interferon upon activation, whether interferons could be predominantly responsible for the upregulation of CXCR3 on primary human monocytes was investigated. Primary human monocytes from three healthy donors and the THP-1 monocyte cell line were treated with varying concentrations of either interferon alpha (IFNα) or interferon gamma (IFNγ) and investigated the impact on CXCR3 expression. The results revealed no significant changes in CXCR3 expression over 24 hours (see FIG. 13).

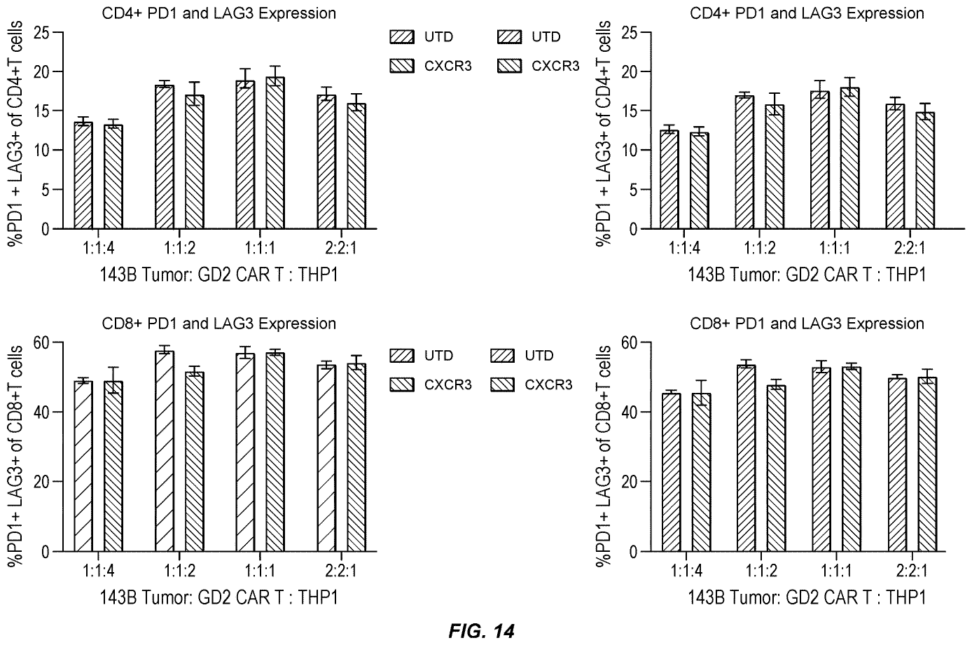

Whether direct interaction of CXCR3-expressing monocytes with GD2 CAR-T cells would have an impact on CAR-T function also was investigated. The CXCR3 THP-1 cells were cultured with GD2 CAR-Ts in the presence or absence of GD2+ 143B osteosarcoma tumor cells at various ratios for 22 hours. No significant changes in the expression of activation markers (CD25, CD69) or exhaustion markers (PD1, Lag3, Tim3) were observed by flow cytometry between the GD2 CAR T cells co-cultured with UTD versus CXCR3-overexpressing THP-1 cells at the ratios tested. Pertinent data are presented in FIG. 14.

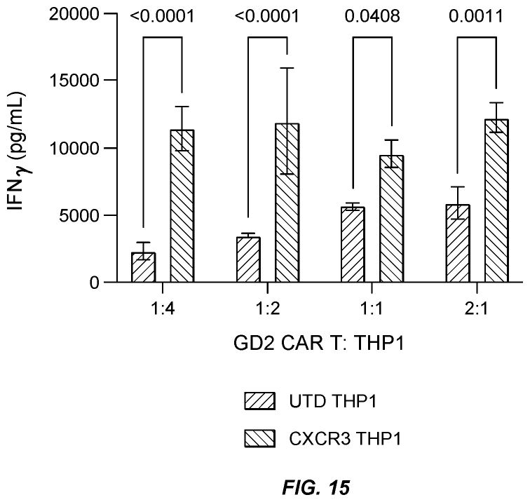

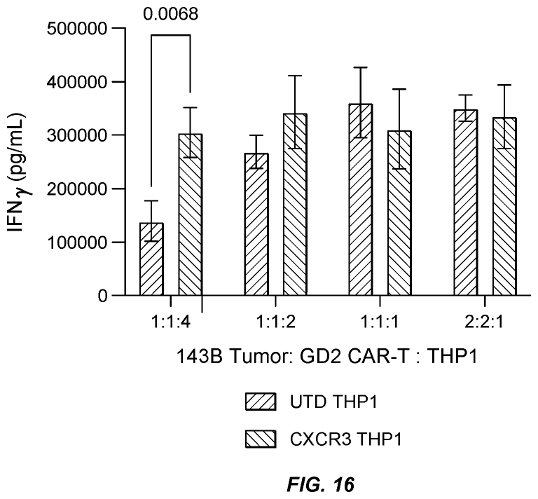

Control untransduced (UTD) THP1 cells cultured with GD2 CAR-T cells reduced IFNγ production by GD2 CAR-T cells in a dose-dependent manner, while co-culture with CXCR3 THP1 cells resulted in enhanced IFNγ production compared to UTD THP1s at all ratios tested (see FIG. 15). These data suggest a role for monocyte-derived CXCR3 to impact CAR-T cell function. Also, the study revealed that in the presence of GD2+ 143B osteosarcoma tumor cells, CXCR3 THP1s stimulated significantly higher IFNγ by GD2 CAR T cells at the highest myeloid ratios (see FIG. 16).

### REFERENCES

1. Louis, D. N., et al. The 2016 World Health Organization Classification of Tumors of the Central Nervous System: a summary. Acta Neuropathol 131, 803-820 (2016).
2. Perkins, S. M., Shinohara, E. T., DeWees, T. & Frangoul, H. Outcome for children with metastatic solid tumors over the last four decades. PLoS One 9, e100396 (2014).
3. Ceschel, S., et al. Survival after relapse in children with solid tumors: a follow-up study from the Italian off-therapy registry. Pediatr Blood Cancer 47, 560-566 (2006).
4. Meyers, P. A., et al. Osteosarcoma: the addition of muramyl tripeptide to chemotherapy improves overall survival—a report from the Children's Oncology Group. J Clin Oncol 26, 633-638 (2008).
5. Cooney, T., et al. Contemporary survival endpoints: an International Diffuse Intrinsic Pontine Glioma Registry study. Neuro Oncol 19, 12791280 (2017).
6. Fisher, P. G., et al. A clinicopathologic reappraisal of brain stem tumor classification. Identification of pilocystic astrocytoma and fibrillary astrocytoma as distinct entities. Cancer 89, 1569-1576 (2000).
7. Chou, A. J., et al. Treatment of osteosarcoma at first recurrence after contemporary therapy: the Memorial Sloan-Kettering Cancer Center experience. Cancer 104, 2214-2221 (2005).
8. Yu, A. L., et al. Anti-GD2 antibody with GM-CSF, interleukin-2, and isotretinoin for neuroblastoma. N Engl J Med 363, 1324-1334 (2010).
9. Pule, M. A., et al. Virus-specific T cells engineered to coexpress tumor specific receptors: persistence and antitumor activity in individuals with neuroblastoma. Nat Med 14, 1264-1270 (2008).
10. Louis, C. U., et al. Antitumor activity and long-term fate of chimeric antigen receptor-positive T cells in patients with neuroblastoma. Blood 118, 6050-6056 (2011).
11. Heczey, A., et al. CAR T Cells Administered in Combination with Lymphodepletion and PD-1 Inhibition to Patients with Neuroblastoma. Mol Ther 25, 2214-2224 (2017).
12. Singh, N., Perazzelli, J., Grupp, S. A. & Barrett, D. M. Early memory phenotypes drive T cell proliferation in patients with pediatric malignancies. Science Translational Medicine 8, 320ra323-320ra323 (2016).
13. Fraietta, J A., et al. Determinants of response and resistance to CD19 chimeric antigen receptor (CAR) T cell therapy of chronic lymphocytic leukemia. Nat Med 24, 563-571 (2018).
14. Das, R. K., Vernau, L., Grupp, S. A. & Barrett, D. M. Naïve T-cell Deficits at Diagnosis and after Chemotherapy Impair Cell Therapy Potential in Pediatric Cancers. Cancer Discov 9, 492-499 (2019).
15. Deng, Q., et al. Characteristics of anti-CD19 CAR T cell infusion products associated with efficacy and toxicity in patients with large B cell lymphomas. Nat Med (2020).
16. Finney, O. C., et al. CD19 CAR T cell product and disease attributes predict leukemia remission durability. J Clin Invest 129, 2123-2132 (2019).
17. Kaczanowska and Beury et al. Genetically engineered myeloid cells rebalance the core immune suppression program in metastasis. Cell, 184(8): 2033-2052 (2021).
18. Long, A. H., et al. 4-1BB costimulation ameliorates T cell exhaustion induced by tonic signaling of chimeric antigen receptors. Nat Med 21, 581-590 (2015).
19. Gargett, T., et al. GD2-specific CAR T Cells Undergo Potent Activation and Deletion Following Antigen Encounter but can be Protected From Activation-induced Cell Death by PD-1 Blockade. Molecular Therapy 24, 1135-1149 (2016).
20. Majzner, R. G., et al. GD2-CAR T cell therapy for H3K27M-mutated diffuse midline gliomas. Nature (2022).
21. Heiner, J. P., et al. Localization of GD2-specific monoclonal antibody 3F8 in human osteosarcoma. Cancer Res 47, 5377-5381 (1987).
22. Helfand, S. C., Hank, J. A., Gan, J. & Sondel, P. M. Lysis of human tumor cell lines by canine complement plus monoclonal antiganglioside antibodies or natural canine xenoantibodies. Cell Immunol 167, 99-107 (1996).
23. Murray, J. L., et al. Phase I trial of murine monoclonal antibody 14G2a administered by prolonged intravenous infusion in patients with neuroectodermal tumors. J Clin Oncol 12, 184-193 (1994).
24. Long, A. H., et al. Reduction of MDSCs with All-trans Retinoic Acid Improves CAR Therapy Efficacy for Sarcomas. Cancer Immunol Res 4, 869880 (2016).
25. Stroncek, D. F., et al. Elutriated lymphocytes for manufacturing chimeric antigen receptor T cells. J Transl Med 15, 59 (2017).
26. Good, Z., et al. Single-cell developmental classification of B cell precursor acute lymphoblastic leukemia at diagnosis reveals predictors of relapse. Nature medicine 24, 474-483 (2018).
27. Sahaf, B., et al. Immune Profiling Mass Cytometry Assay Harmonization: Multicenter Experience from CIMAC-CIDC. Clin Cancer Res 27, 5062-5071 (2021).
28. Finck, R., et al. Normalization of mass cytometry data with bead standards. Cytometry A 83, 483-494 (2013).
29. Lun, A. T. L., Richard, A. C. & Marioni, J. C. Testing for differential abundance in mass cytometry data. Nat Methods 14, 707-709 (2017).
30. Nowicka, M., et al. CyTOF workflow: differential discovery in high throughput high-dimensional cytometry datasets. F1000Res 6, 748 (2017).
31. Van Gassen, S., et al. FlowSOM: Using self-organizing maps for visualization and interpretation of cytometry data. Cytometry A 87, 636645 (2015).
32. Bates, D., Machler, M., Bolker, B. & Walker, S. Fitting Linear Mixed-Effects Models using lme4. arXiv:1406.5823 (2014).
33. Angerer, P., et al. destiny: diffusion maps for large-scale single-cell data in R. Bioinformatics 32, 1241-1243 (2016).
34. Liaw, A. & Wiener, M. C. Classification and Regression by randomForest. (2007).
35. Kuhn, M. Building Predictive Models in R Using the caret Package. Journal of Statistical Software 28, 1-26 (2008).
36. Therneau, T. M. & Atkinson, E. J. An Introduction to Recursive Partitioning Using the RPART Routines. (2015).
37. Milborrow, S. rpart.plot: Plot rpart Models. An Enhanced Version of plot.rpart. (2016).
38. Dobin, A., et al. STAR: ultrafast universal RNA-seq aligner. Bioinformatics 29, 15-21 (2013).
39. Wang, L., Wang, S. & Li, W. RSeQC: quality control of RNA-seq experiments. Bioinformatics 28, 2184-2185 (2012).
40. Patro, R., Duggal, G., Love, M. I., Irizarry, R. A. & Kingsford, C. Salmon provides fast and bias-aware quantification of transcript expression. Nat Methods 14, 417-419 (2017).
41. Love, M. I., Huber, W. & Anders, S. Moderated estimation of fold change and dispersion for RNA-seq data with DESeq2. Genome Biol 15, 550 (2014).
42. Yu, G., Wang, L. G., Han, Y. & He, Q. Y. clusterProfiler: an R package for comparing biological themes among gene clusters. OMICS 16, 284-287 (2012).
43. Liberzon, A., et al. The Molecular Signatures Database (MSigDB) hallmark gene set collection. Cell Syst 1, 417-425 (2015).
44. Chen, B., Khodadoust, M. S., Liu, C. L., Newman, A. M. & Alizadeh, A. A. Profiling Tumor Infiltrating Immune Cells with CIBERSORT. Methods Mol Biol 1711, 243-259 (2018).
45. Sturm, G., Finotello, F. & List, M. Immunedeconv: An R Package for Unified Access to Computational Methods for Estimating Immune Cell Fractions from Bulk RNA-Sequencing Data. Methods Mol Biol 2120, 223232 (2020).
46. Corces, M R., et al. An improved ATAC-seq protocol reduces background and enables interrogation of frozen tissues. Nat Methods 14, 959-962 (2017).
47. Gyurdieva, A., et al. Biomarker correlates with response to NY-ESO-1 TCR T cells in patients with synovial sarcoma. Nature Communications 13, 5296 (2022).
48. Piedra-Quintero, Z. L., Wilson, Z., Nava, P. & Guerau-de-Arellano, M. CD38: An Immunomodulatory Molecule in Inflammation and Autoimmunity. Frontiers in Immunology 11(2020).
49. Lynn, R. C., et al. c-Jun overexpression in CAR T cells induces exhaustion resistance. Nature 576, 293-300 (2019).
50. Canale, F. P., et al. CD39 Expression Defines Cell Exhaustion in Tumor Infiltrating CD8(+) T Cells. Cancer Res 78, 115-128 (2018).
51. Gupta, P. K., et al. CD39 Expression Identifies Terminally Exhausted CD8+ T Cells. PLoS Pathog 11, e1005177 (2015).
52. Mulder, K., et al. Cross-tissue single-cell landscape of human monocytes and macrophages in health and disease. Immunity 54, 1883-1900.e1885 (2021).
53. Veglia, F., et al. Analysis of classical neutrophils and polymorphonuclear myeloid-derived suppressor cells in cancer patients and tumor-bearing mice. Journal of Experimental Medicine 218(2021).
54. Loeuillard, E., et al. Targeting tumor-associated macrophages and granulocytic myeloid-derived suppressor cells augments PD-1 blockade in cholangiocarcinoma. The Journal of Clinical Investigation 130, 5380-5396 (2020).
55. Zhang, Y., et al. Single-cell analyses reveal key immune cell subsets associated with response to PD-L1 blockade in triple-negative breast cancer. Cancer Cell 39, 1578-1593.e1578 (2021).
56. Dower, K., Ellis, D. K., Saraf, K., Jelinsky, S. A. & Lin, L. L. Innate immune responses to TREM-1 activation: overlap, divergence, and positive and negative cross-talk with bacterial lipopolysaccharide. J Immunol 180, 3520-3534 (2008).
57. Friedman, A. D. Transcriptional control of granulocyte and monocyte development. Oncogene 26, 6816-6828 (2007).
58. Zabuawala, T., et al. An Ets2-Driven Transcriptional Program in Tumor Associated Macrophages Promotes Tumor Metastasis. Cancer Research 70, 1323-1333 (2010).
59. Padgett, L. E., et al. Interplay of Monocytes and T Lymphocytes in COVID19 Severity. bioRxiv, 2020.2007.2017.209304 (2020).
60. Maude, S. L., Teachey, D. T., Porter, D. L. & Grupp, S. A. CD19-targeted chimeric antigen receptor T-cell therapy for acute lymphoblastic leukemia. Blood 125, 4017-4023 (2015).
61. Lee, D. W., et al. T cells expressing CD19 chimeric antigen receptors for acute lymphoblastic leukaemia in children and young adults: a phase 1 dose-escalation trial. Lancet 385, 517-528 (2015).
62. Fry, T. J., et al. CD22-targeted CAR T cells induce remission in B-ALL that is naive or resistant to CD19-targeted CAR immunotherapy. Nature Medicine 24, 20 (2017).
63. Ramakrishna, S., Barsan, V. & Mackall, C. Prospects and challenges for use of CAR T cell therapies in solid tumors. Expert Opin Biol Ther 20, 503516 (2020).
64. Chen, G. M., et al. Integrative Bulk and Single-Cell Profiling of Premanufacture T-cell Populations Reveals Factors Mediating Long-Term Persistence of CAR T-cell Therapy. Cancer Discov 11, 2186-2199 (2021).
65. Abron, J. D., et al. Differential role of CXCR3 in inflammation and colorectal cancer. Oncotarget 9, 17928-17936 (2018).
66. Butler, K. L., Clancy-Thompson, E. & Mullins, D. W. CXCR3+ monocytes/macrophages are required for establishment of pulmonary metastases. Scientific Reports 7, 45593 (2017).
67. Russo, E., Santoni, A. & Bernardini, G. Tumor inhibition or tumor promotion? The duplicity of CXCR3 in cancer. Journal of Leukocyte Biology 108, 673-685 (2020).

TABLE 1
Patient Demographics and Baseline Characteristics
of Patients Treated with GD2 CAR

| | | Number of |
|---|---|---|
| Characteristic | | patients (n = 15) |
| Age | Median (range) | 17 (8-28) years |
| Sex | Female/Male | 3/12 |
| Race | White | 9 |
| | African American | 3 |
| | Asian | 1 |
| | Hispanic | 1 |
| | Multiple Race | 1 |
| Tumor Type | Osteosarcoma | 12 |
| | Neuroblastoma | 3 |
| Prior Therapies | Surgery | 14 |
| | Chemotherapy | 15 |
| | Radiation | 6 |
| | Immunotherapy/Targeted Therapy | 2 |
| | Bone marrow transplant | 2 |

TABLE 2
Patient Enrollment and Duration of Treatment

| | | Dose Level | Day 28 | Best | Reason Off Treatment CIDC |
|---|---|---|---|---|---|
| Patient | Diagnosis | (Cells/kg) | Response | Response | (Days Since CAR-T) |
| 1 | Osteosarcoma | 1 × 10⁵ | SD | SD (26) | Disease Progression On Study (62) |
| 2 | Osteosarcoma | 1 × 10⁵ | N/A | N/A | Disease Progression before Treatment |
| 3 | Osteosarcoma | 1 × 10⁵ | N/A | PD (12) | Switched to Alternative Treatment (12) |
| 4 | Osteosarcoma | 1 × 10⁵ | PD | PD (25) | Disease Progression On Study (25) |
| 5 | Osteosarcoma | 1 × 10⁵ | SD | PD | Disease Progression On Study (60) |
| 6 | Osteosarcoma | 1 × 10⁶ | SD | SD (26) | Switched to Alternative Treatment (55) |
| 7 | Neuroblastoma | 1 × 10⁶ | SD | SD (27) | Switched to Alternative Treatment (163) |
| 8 | Osteosarcoma | 1 × 10⁶ | SD | SD (28) | Switched to Alternative Treatment (70) |
| 9 | Neuroblastoma | 3 × 10⁶ | SD | SD (26) | Switched to Alternative Treatment (76) |
| 10 | Osteosarcoma | 3 × 10⁶ | SD | SD (28) | Disease Progression On Study (60) |
| 11 | Neuroblastoma | 3 × 10⁶ | N/A | N/A | Disease Progression before Treatment |
| 12 | Osteosarcoma | 3 × 10⁵ | SD | SD (59) | Switched to Alternative Treatment (89) |
| 13 | Osteosarcoma | 1 × 10⁷ | SD | SD (27) | Elective Withdrawal (165) |
| 14 | Osteosarcoma | 1 × 10⁷ | SD | SD (30) | Switched to Alternative Treatment (105) |
| 15 | Osteosarcoma | 1 × 10⁷ | PD | PD (27) | Switched to Alternative Treatment (40) |

TABLE 3
AF

| | | | Time | Duration | Duration | Duration | |
|---|---|---|---|---|---|---|---|
| | | | to | of | of | of | |
| | | | Onset | Symptoms | Symptoms | Symptoms | |
| | | No. of | (days, | (days, | (days, | (days, | |
| System | Grade | Patients | range) | mean) | median) | max) | Adverse Events |
| Cardiovascular | 1 | 9 | 1-27 | 3.0 | 2 | 18 | Sinus tachycardia, hypertension, hypotension, tachycardia, |
| | 2 | 4 | 1-28 | 8.5 | 5 | 29 | elevated D-dimer, thromboembolic event |
| | NA | 1 | 1 | 0.0 | 0 | 0 | |
| CRS | 1 | 3 | 6-10 | 3.7 | 3 | 7 | Cytokine release syndrome |
| | 2 | 1 | ? | 6.0 | 6 | 6 | |
| Gastroenterological | 1 | 11 | 1-41 | 3.5 | 2 | 14 | GERD, constipation, nausea, toothache, vomiting, dysphagia, |
| | 2 | 4 | 9-23 | 3.8 | 4 | 6 | oral pain and mucositis, rectal pain and mucositis, diarrhea, |
| | | | | | | | abdominal pain, ascites, dyspepsia |
| General | 1 | 12 | 1-22 | 4.7 | 1 | 53 | Fever, hypothermia, weight loss, tumor pain, general pain, |
| | 2 | 8 | 1-17 | 0.4 | 0 | 1 | fatigue, peripheral edema, gait disturbance, chest pain (non- |
| | 3 | 2 | 11-12 | 0.0 | 0 | 0 | cardiac), localized edema, elevated c-reactive protein, spitting |
| Hematologic | 1 | 14 | 1-64 | 6.2 | 3 | 40 | Neutropenia, thrombocytopenia, lymphopenia, anemia, |
| | 2 | 15 | 1-97 | 5.2 | 2 | 43 | leukopenia, prolonged activated PTT, febrile neutropenia |
| | 3 | 15 | 1-72 | 4.4 | 1 | 36 | |
| | 4 | 15 | 1-72 | 6.8 | 4 | 60 | |
| Hepatobiliary | 1 | 11 | 2-28 | 12.6 | 3 | 53 | Elevated ALT, elevated AST, elevated ALP, elevated CPK, |
| | 2 | 3 | 2-9 | 3.3 | 3 | 6 | elevated bilirubin |
| | 3 | 4 | 8-27 | 17.3 | 11 | 56 | |
| | 4 | 3 | 11-12 | 2.0 | 2 | 2 | |
| Infection | 1 | 1 | 26 | N/R | N/R | N/R | C. difficile, Streptococcus Group C, S. epidermidis, EBVCA, |
| | 2 | 3 | 7-18 | 10.0 | 8 | 23 | Hordelum (eye) |
| Metabolic | 1 | 15 | 1-29 | 2.4 | 2 | 27 | Hypercalcemia, hyperuricemia, hyperglycemia, hypocalcemia, |
| | 2 | 8 | 1-28 | 5.3 | 1 | 69 | hypoalbuminemia, hypoglycemia, hyponatremia, |
| | 3 | 4 | 7-14 | 1.6 | 1 | 4 | hypophosphatemia, hypokalemia, hypomagnesemia, |
| | | | | | | | dehydration, |
| Musculoskeletal | 1 | 5 | 1-15 | 7.3 | 6 | 17 | Back pain, buttocks pain, chest wall pain, extremity pain, |
| | 2 | 2 | 7-19 | 5.0 | 5 | 5 | cramping |
| | 3 | 1 | 18 | N/R | N/R | N/R | |
| Neurologic, other | 1 | 8 | 1-19 | 0.9 | 1 | 3 | Akathisia, dizziness, dysgeusia, headache, paresthesia, |
| | 2 | 2 | 7-10 | 1.0 | 1 | 1 | peripheral sensory neuropathy |
| Ophthalmic | 1 | 1 | 8 | 0.0 | 0 | 0 | Eye pain |
| Psychiatric | 1 | 3 | 1-18 | 29.3 | 12 | 93 | Anxiety, depression, hallucinations, insomnia |
| | 2 | 1 | 23 | 31.0 | 31 | 31 | |
| Renal | 1 | 6 | 1-28 | 2.6 | 1 | 11 | Hematuria, proteinuria, urinary frequency, urinary incontinence, |
| | 2 | 2 | 1-19 | 2.5 | 1 | 7 | urinary tract pain, urinary retention |
| Reproductive/ | 1 | 2 | 11-15 | 7.5 | 8 | 8 | Genital edema, vaginal discharge, vaginal pruritus |
| Breast | | | | | | | |
| Respiratory | 1 | 8 | 2-64 | 3.4 | 2 | 13 | Atelectasis, cough, decreased carbon monoxide diffusing |
| | 2 | 2 | 2-26 | 1.7 | 2 | 3 | capacity, dyspnea, epistaxis, hypoxia, nasal congestion, pleural |
| | 3 | 1 | 10-11 | 3.0 | 3 | 5 | effusion, pneumothorax, sore throat, tachypnea, tracheal |
| | 4 | 1 | 9 | 1.0 | 1 | 1 | mucositis |
| | 5 | 1 | 15 | 1.0 | 1 | 1 | |
| | NA | 1 | 22 | 9.0 | 9 | 9 | |
| Skin | 1 | 4 | 1-19 | 3.3 | 3 | 8 | Blistering, dry skin, erythema, pruritus |
| | 2 | 1 | 8 | 23.0 | 23 | 23 | |

URLs and other internet links appearing herein have been disabled to prevent them from printing as active links, in accordance with pertinent regulations. The content can be accessed by manually reconstructing the links in a suitable web browser. In particular “[dot]” can be replaced with “.”.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for predicting the susceptibility of a patient suffering from a cancer or a myeloid-mediated disease to immunotherapy, the method comprising
(a) obtaining myeloid cells from the patient prior to treatment and
(b) assaying the myeloid cells for the expression of at least CXCR3 within the myeloid cells,
wherein elevated CXCR3 expression, or a CXCR3 positive population of myeloid cells expressing other markers including but not limited to CX3CR1 or CD16 or CCR7, within the myeloid cells is predictive of patients that will be responsive to cell-based immunotherapy or mark a poorer prognosis inflammatory state.

2. A method for treating a patient suffering from a cancer or a myeloid-mediated disease with cell-based immunotherapy, the method comprising
(a) obtaining myeloid cells from the patient prior to treatment
(b) assaying the myeloid cells for the expression of at least CXCR3 within the myeloid cells,
(c) administering cell-based immunotherapy to the patient when the result of the assay from (b) reveals elevated CXCR3 expression or a CXCR3 positive population of myeloid cells expressing other markers including but not limited to CX3CR1 or CD16 or CCR7, within the myeloid cells.

3. The method of claim 2, wherein the patient suffers from a cancer.

4. The method of claim 2, wherein the patient suffers from a cancer selected from the group consisting of sarcomas, neuroblastoma, neuroendocrine tumors, breast cancer, pancreatic adenocarcinoma, ovarian cancer, and melanoma.

5. The method of claim 2, wherein the patient suffers from a cancer comprising a solid tumor.

6. The method of claim 2, wherein the patient suffers from a myeloid-mediated disease selected from the group consisting of autoimmune or inflammatory disorders, degenerative disorders, and infectious diseases.

7. The method of claim 6, wherein an autoimmune or inflammatory disorder comprises inflammatory bowel disease (IBD), rheumatoid arthritis, plaque psoriasis, lupus, and graft versus host disease (GVHD).

8. The method of claim 6, wherein a degenerative disorder comprises neurodegeneration.

9. The method of claim 6, wherein the degenerative disorder comprises Alzheimer's Disease.

10. The method of claim 2, wherein the patient suffers from an inflammatory disease or disorder.

11. The method of claim 10, wherein the inflammatory disease or disorder comprises sepsis, or atherosclerosis.

12. The method of claim 2, wherein the patient suffers from an infectious disease, such as COVID.

13. The method of claim 2, wherein the immunotherapy comprises the administration of a chimeric antigen receptor T cell (CAR-T cell), a genetically engineered myeloid cells (GEMy), transgenic TCR T cells and TIL therapy, immune checkpoint blockade, or a combination thereof, to the patient.

14. The method of claim 13, wherein the immunotherapy comprises administering a CAR-T cell, which targets CD19, BCMA GD2, B87H3, FGFR4, CD22, Her2, or a combination thereof.

15. The method of claim 13, wherein the immunotherapy comprises administering a GEMy that secretes “interleukin-12” (IL12), soluble “triggering receptor expressed on myeloid cells 2” (TREM2), “cluster of differentiation 40 ligand” (CD40L), “interleukin 6 decoy receptor” (IL6DR), IL1BRA or GEMesy or GEMys expressing Hyal or a combination thereof.

16. The method of claim 2, wherein the immunotherapy comprises administering a checkpoint inhibitor to the patient.

17. The method of claim 2, wherein the myeloid cells are obtained from apheresis or whole blood product obtained from the patient, which optionally is enriched for monocytes, macrophages, and dendritic cells (DC).

18. The method of claim 2, wherein the myeloid cells are further assayed for the expression of CD33.

19. The method of claim 2, wherein the myeloid cells are further assayed for the expression of CD169.

20. The method of claim 2, wherein the assay for expression comprises mass spectrometry, flow cytometry, or ELISA.

21. The method of claim 20, wherein the assay for expression comprises “cytometry by time of flight” (CyTOF).

22. A GEMy cell comprising a myeloid cell comprising an exogenous genetic expression construct comprising a nucleic sequence encoding a CXCR3 protein.

23. (canceled)

24. A GEMy cell comprising a myeloid cell genetically modified to reduce or eliminate production of a CXCR3 protein.

25-56. (canceled)


SEQUENCE LISTING (11 sequences)

SEQ ID NO: 1 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 2 - protein, 415 aa, Homo sapiens
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 3 - protein, 267 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQGSSSGSGCGCCSCAWAAPTREGSRGSHRLPAGIHPGLRPQRPPTRACEAGIRAPLSPI

SEQ ID NO: 4 - RNA, 1104 nt, Homo sapiens
  source 1..1104: mol_type = genomic RNA
ccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttg

SEQ ID NO: 5 - RNA, 767 nt, Homo sapiens
  source 1..767: mol_type = genomic RNA
ccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggggtcaagttccgggagcggatgtggatgctgctcttgcgcctgggctgccccaaccagagagggctccagaggcagccatcgtcttcccgccgggattcatcctggtctgagacctcagaggcctcctactcgggcttg

SEQ ID NO: 6 - DNA, 22 bp, Homo sapiens
  source 1..22: mol_type = genomic DNA
gaaagctgactgcccctatttg

SEQ ID NO: 7 - DNA, 22 bp, Homo sapiens
  source 1..22: mol_type = genomic DNA
gagaggaagtgctgggaacaat

SEQ ID NO: 8 - DNA, 15 bp, Homo sapiens
  source 1..15: mol_type = genomic DNA
ctccccagtctcttt

SEQ ID NO: 9 - DNA, 15 bp, Homo sapiens
  source 1..15: mol_type = genomic DNA
cgcccactccaccct

SEQ ID NO: 10 - DNA, 18 bp, Homo sapiens
  source 1..18: mol_type = genomic DNA
gttctggccctgctggta

SEQ ID NO: 11 - DNA, 19 bp, Homo sapiens
  source 1..19: mol_type = genomic DNA
caagatcagagtgaagttc